(19) United States
(12) Patent Application Publication
(10) Pub. No.: US 2023/0301121 A1
(43) Pub. Date: Sep. 21, 2023

(54) PHOTOELECTRIC CONVERSION ELEMENT, IMAGING ELEMENT, OPTICAL SENSOR, AND COMPOUND

(71) Applicant: FUJIFILM Corporation, Tokyo (JP)

(72) Inventors: Kazuhei KANEKO, Kanagawa (JP); Hiroki Sugiura, Kanagawa (JP)

(73) Assignee: FUJIFILM Corporation, Tokyo (JP)

(21) Appl. No.: 18/167,875

(22) Filed: Feb. 12, 2023

(30) Foreign Application Priority Data
Feb. 15, 2022 (JP) 2022-021472

(51) Int. Cl. H10K 30/20; H10K 85/60; H10K 85/20

(52) U.S. Cl. CPC H10K 30/20; H10K 85/211; H10K 85/652; H10K 85/654; H10K 85/657; H10K 85/6572; H10K 85/6576; H10K 39/32

(57) ABSTRACT

A photoelectric conversion element having a high photoelectric conversion efficiency in a visible light region (particularly, a wavelength range of 450 to 650 nm) even after being subjected to heat treatment (annealing) is provided. In addition, an imaging element, an optical sensor, and a compound are provided.

The photoelectric conversion element includes, in the following order, a conductive film, a photoelectric conversion film, and a transparent conductive film, and the photoelectric conversion film contains a compound represented by Formula (1).

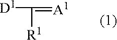

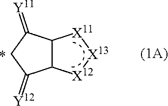

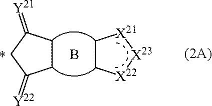

### CROSS-REFERENCE TO RELATED APPLICATIONS

The present application claims priority under 35 U.S.C. § 119 to Japanese Patent Application No. 2022-021472, filed on Feb. 15, 2022. The above application is hereby expressly incorporated by reference, in its entirety, into the present application.

### BACKGROUND OF THE INVENTION

### 1. Field of the Invention

The present invention relates to a photoelectric conversion element, an imaging element, an optical sensor, and a compound.

### 2. Description of the Related Art

In recent years, the development of an element (for example, an imaging element) having a photoelectric conversion film has been progressing.

For example, it is disclosed in WO2020/013246A that a compound described below is used as a material applied to a photoelectric conversion element.

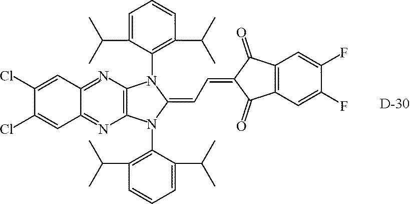 [SMILES: CC(C)c1cccc(C(C)C)c1N1C(=CC=C2C(=O)c3cc(F)c(F)cc3C2=O)N(c2c(C(C)C)cccc2C(C)C)c2nc3cc(Cl)c(Cl)cc3nc21]

### SUMMARY OF THE INVENTION

In recent years, along with the demand for improving the performance of imaging elements, optical sensors, and the like, further improvements are required for various characteristics required for photoelectric conversion elements used therein.

The present inventors have produced and examined a photoelectric conversion element obtained by using the compound disclosed in WO2020/013246A, and have found that the photoelectric conversion element that has been heat-treated (annealed) have had a photoelectric conversion efficiency in a visible light region (particularly, a wavelength range of 450 to 650 nm), which does not satisfy a desired required level, and it was clarified that the photoelectric conversion element has been required to be further improved. That is, it was clarified that there is room for studying a photoelectric conversion element having a high photoelectric conversion efficiency in a visible light region (particularly, a wavelength range of 450 to 650 nm) even after being subjected to heat treatment (annealing).

Thus, an object of the present invention is to provide a photoelectric conversion element having a high photoelectric conversion efficiency in a visible light region (particularly, a wavelength range of 450 to 650 nm) even after being subjected to heat treatment (annealing). Another object of the present invention is to provide an imaging element, an optical sensor, and a compound.

The inventors of the present invention have conducted extensive studies on the above-described problems. As a result, the inventors have found that it is possible to solve the above-described problems by applying the compound having a predetermined structure to the photoelectric conversion film, and have completed the present invention.

[1] A photoelectric conversion element comprising, in the following order:
a conductive film;
a photoelectric conversion film; and
a transparent conductive film,
in which the photoelectric conversion film at least contains a compound represented by Formula (1).

The photoelectric conversion element according to [1], in which one of Y¹¹ or Y¹² represents an oxygen atom or a sulfur atom, and

one of Y²¹ or Y²² represents an oxygen atom or a sulfur atom.

The photoelectric conversion element according to [1], in which one of Y¹¹ or Y¹² represents ═C(CN)₂, and

one of Y²¹ or Y²² represents ═C(CN)₂.

The photoelectric conversion element according to any one of [1] to [3], in which A¹ represents a group represented by Formula (1Aa-1).

The photoelectric conversion element according to any one of [1] to [4], in which D¹ represents a group represented by any one of Formula (1D) or Formula (2D).

The photoelectric conversion element according to [5], in which D¹ represents a group represented by Formula (1D).

The photoelectric conversion element according to [6], in which the group represented by Formula (1D) represents a group represented by Formula (3D).

The photoelectric conversion element according to [6], in which the group represented by Formula (1D) represents a group represented by Formula (4D).

The photoelectric conversion element according to [6], in which the group represented by Formula (1D) is a group represented by Formula (5D).

The photoelectric conversion element according to any one of [5] to [9], in which structures of R^{d11} and R^{d12} are different from each other.

The photoelectric conversion element according to any one of [5] to [10], in which one of R^{d11} or R^{d12} represents an alkyl group which may have a substituent, and the other represents a group represented by Formula (X).

The photoelectric conversion element according to [11], in which the group represented by Formula (X) represents a group represented by Formula (ZB).

The photoelectric conversion element according to [5], in which D¹ represents a group represented by Formula (2D), and

the group represented by Formula (2D) represents a group represented by Formula (6D).

The photoelectric conversion element according to [13], in which Formula (6D) satisfies at least one or more following requirements,
a requirement X1: R^{d22} represents an aromatic ring group which may have a substituent,
a requirement X2: X^{d21} represents -C(R^{L24})(R^{L25})-, and R^{L24} and R^{L25} are bonded to each other to form a ring, and
a requirement X3: any one or more of R^{d61} and R^{d62}, R^{d62} and R^{d63}, and R^{d63} and R^{d64} are bonded to each other to form a ring.

The photoelectric conversion element according to any one of [1] to [14], in which the photoelectric conversion film further contains a n-type semiconductor.

The photoelectric conversion element according to [15], in which the n-type semiconductor includes fullerenes selected from the group consisting of a fullerene and a derivative thereof.

The photoelectric conversion element according to any one of [1] to [16], in which the photoelectric conversion film further contains a p-type semiconductor.

The photoelectric conversion element according to any one of [1] to [17], further comprising one or more interlayers between the conductive film and the transparent conductive film, in addition to the photoelectric conversion film.

An imaging element comprising the photoelectric conversion element according to any one of [1] to [18].

An optical sensor comprising the photoelectric conversion element according to any one of [1] to [19].

A compound represented by Formula (1).

The compound according to [21], in which one of Y¹¹ or Y¹² represents an oxygen atom or a sulfur atom, and

one of Y²¹ or Y²² represents an oxygen atom or a sulfur atom.

The compound according to [21], in which one of Y¹¹ or Y¹² represents ═C(CN)₂, and

one of Y²¹ or Y²² represents ═C(CN)₂.

The compound according to any one of [21] to [23], in which A¹ represents a group represented by Formula (1Aa-1).

The compound according to any one of [21] to [24], in which D¹ represents a group represented by any one of Formula (1D) or Formula (2D).

The compound according to [25], in which D¹ represents a group represented by Formula (1D).

The compound according to [26], in which the group represented by Formula (1D) represents a group represented by Formula (3D).

The compound according to [26], in which the group represented by Formula (1D) represents a group represented by Formula (4D).

The compound according to [26], in which the group represented by Formula (1D) is a group represented by Formula (5D).

The compound according to any one of [25] to [29], in which structures of R^{d11} and R^{d12} are different from each other.

The compound according to any one of [25] to [30], in which one of R^{d11} or R^{d12} represents an alkyl group which may have a substituent, and the other represents a group represented by Formula (X).

The compound according to [31], in which the group represented by Formula (X) represents a group represented by Formula (ZB).

The compound according to [25], in which D¹ represents a group represented by Formula (2D), and

the group represented by Formula (2D) represents a group represented by Formula (6D).

The compound according to [33], in which Formula (6D) satisfies at least one or more following requirements,
a requirement X1: R^{d22} represents an aromatic ring group which may have a substituent,
a requirement X2: X^{d21} represents -C(R^{L24})(R^{L25})-_{,} and R^{L24} and R^{L25} are bonded to each other to form a ring, and
a requirement X3: any one or more of R^{d61} and R^{d62}, R^{d62} and R^{d63}, and R^{d63} and R^{d64} are bonded to each other to form a ring.

According to the present invention, it is possible to provide the photoelectric conversion element having a high photoelectric conversion efficiency in a visible light region (particularly, a wavelength range of 450 to 650 nm) even after being subjected to heat treatment (annealing). In addition, according to the present invention, it is possible to provide the imaging element, the optical sensor, and the compound.

### BRIEF DESCRIPTION OF THE DRAWINGS

FIG. 1 is a schematic cross-sectional view illustrating a configuration example of a photoelectric conversion element.

FIG. 2 is a schematic cross-sectional view illustrating a configuration example of the photoelectric conversion element.

### DESCRIPTION OF THE PREFERRED EMBODIMENTS

Hereinafter, suitable embodiments of a photoelectric conversion element of the present invention will be described.

In the present specification, the numerical range represented by “to” means a range including numerical values denoted before and after “to” as a lower limit value and an upper limit value.

In the present specification, in a case where there are plural substituents, linking groups, and the like (hereinafter, referred to as “substituents and the like”) represented by specific symbols, or a case where a plurality of substituents and the like are specified all together, each of the substituents and the like may be the same or may be different from each other. This also applies to a case of specifying the number of substituents and the like.

In the present specification, a hydrogen atom may be a light hydrogen atom (an ordinary hydrogen atom) or a deuterium atom (for example, a double hydrogen atom and the like).

In the present specification, examples of the halogen atom include a fluorine atom, a chlorine atom, a bromine atom, and an iodine atom.

In the present specification, a “substituent” includes a group exemplified by a substituent W described later, unless otherwise specified.

### Substituent W

A substituent W in the present specification will be described below.

Examples of the substituent W include a halogen atom (for example, a fluorine atom, a chlorine atom, a bromine atom, an iodine atom, and the like), an alkyl group (including a cycloalkyl group, a bicycloalkyl group, and a tricycloalkyl group), an alkenyl group (including a cycloalkenyl group and a bicycloalkenyl group), an alkynyl group, an aryl group, a heteroaryl group (may also be referred to as a heterocyclic group), a cyano group, a nitro group, an alkoxy group, an aryloxy group, a silyloxy group, a heterocyclic oxy group, an acyloxy group, a carbamoyloxy group, an alkoxycarbonyloxy group, an aryloxycarbonyloxy group, a secondary or tertiary amino group (including an anilino group), an alkylthio group, an arylthio group, a heterocyclic thio group, an alkyl or an arylsulfinyl group, an alkyl or an arylsulfonyl group, an acyl group, an aryloxycarbonyl group, an alkoxycarbonyl group, an aryl or a heterocyclic azo group, an imide group, a phosphino group, a phosphinyl group, a phosphinyloxy group, a phosphinylamino group, a phosphono group, a silyl group, a carboxy group, a phosphoric acid group, a sulfonic acid group, a hydroxy group, a thiol group, an acylamino group, a carbamoyl group, a ureido group, a boronic acid group, and a primary amino group. Each of the above-described groups may further have a substituent (for example, one or more groups of each of the above-described groups), as possible. For example, an alkyl group which may have a substituent is also included as a form of the substituent W.

In addition, in a case where the substituent W has a carbon atom, the number of carbon atoms of the substituent W is, for example, 1 to 20.

The number of atoms other than a hydrogen atom included in the substituent W is, for example, 1 to 30.

In addition, the specific compound preferably does not contain, as a substituent, a carboxy group, a salt of a carboxy group, a salt of a phosphoric acid group, a sulfonic acid group, a salt of a sulfonic acid group, a hydroxy group, a thiol group, an acylamino group, a carbamoyl group, a ureido group, or a boronic acid group (—B(OH)₂) and/or a primary amino group.

In the present specification, unless otherwise specified, the number of carbon atoms of the alkyl group is preferably 1 to 20, more preferably 1 to 12, still more preferably 1 to 10, and particularly preferably 1 to 6.

The alkyl group may be any one of linear, branched, or cyclic.

Examples of the alkyl group include a methyl group, an ethyl group, a n-propyl group, an i-propyl group, a n-butyl group, a t-butyl group, a n-hexyl group, a cyclopentyl group, and the like.

In addition, the alkyl group may be, for example, a cycloalkyl group, a bicycloalkyl group, or a tricycloalkyl group, and may have a cyclic structure thereof as a partial structure.

In the alkyl group which may have a substituent, a substituent which may be contained in the alkyl group is not particularly limited, an example thereof includes the substituent W, and an aryl group (preferably having 6 to 18 carbon atoms, and more preferably having 6 carbon atoms), a heteroaryl group (preferably having 5 to 18 carbon atoms, and more preferably having 5 and 6 carbon atoms), or a halogen atom (preferably a fluorine atom or a chlorine atom) is preferable.

In the present specification, unless otherwise specified, the above-described alkyl group is preferable as an alkyl group moiety in the alkoxy group. The alkyl group moiety in the alkylthio group is preferably the above-described alkyl group.

In the alkoxy group which may have a substituent, the substituent which may be contained in the alkoxy group includes the same examples as the substituent in the alkyl group which may have a substituent. In the alkylthio group which may have a substituent, the substituent which may be contained in the alkylthio group includes the same examples as the substituent in the alkyl group which may have a substituent.

In the present specification, the alkenyl group may be any of linear, branched, or cyclic, unless otherwise specified. The number of carbon atoms of the alkenyl group is preferably 2 to 20, more preferably 2 to 12, still more preferably 2 to 6, and particularly preferably 2 or 3. In the alkenyl group which may have a substituent, the substituent which may be contained in the alkenyl group includes the same examples as the substituent in the alkyl group which may have a substituent.

In the present specification, an alkynyl group may be any of linear, branched, or cyclic, unless otherwise specified. The number of carbon atoms of the alkynyl group is preferably 2 to 20, more preferably 2 to 12, still more preferably 2 to 6, and particularly preferably 2 or 3. In the alkynyl group which may have a substituent, the substituent which may be contained in the alkynyl group includes the same examples as the substituent in the alkyl group which may have a substituent.

In the present specification, unless otherwise specified, examples of a silyl group which may have a substituent include a group represented by —Si(R^{S1})(R^{S2})(R^{S3}). R^{S1}, R^{S2}, and R^{S3} each independently represent a hydrogen atom or a substituent, and preferably represent an alkyl group which may have a substituent, an alkoxy group which may have a substituent, an alkylthio group which may have a substituent, an aryl group which may have a substituent, or a heteroaryl group which may have a substituent.

In the present specification, an aromatic ring may be a monocyclic ring or a polycyclic ring (for example, 2 to 6 rings or the like), unless otherwise specified. The monocyclic aromatic ring is an aromatic ring having only one aromatic ring structure as a ring structure. The polycyclic (for example, 2 to 6 rings or the like) aromatic ring is an aromatic ring formed by a plurality of (for example, 2 to 6 or the like) aromatic ring structures being fused, as a ring structure.

The number of ring member atoms of the aromatic ring is preferably 5 to 15.

The aromatic ring may be an aromatic hydrocarbon ring or an aromatic heterocyclic ring.

In a case where the aromatic ring is an aromatic heterocyclic ring, the number of heteroatoms contained as ring member atoms is, for example, 1 to 10. Examples of the heteroatoms include a nitrogen atom, a sulfur atom, an oxygen atom, a selenium atom, a tellurium atom, a phosphorus atom, a silicon atom, and a boron atom.

Examples of the aromatic hydrocarbon ring include a benzene ring, a naphthalene ring, an anthracene ring, and a phenanthrene ring.

Examples of the aromatic heterocyclic ring include a pyridine ring, a pyrimidine ring, a pyridazine ring, a pyrazine ring, a triazine ring (1,2,3-triazine ring, 1,2,4-triazine ring, 1,3,5-triazine ring, or the like), and a tetrazine ring (1,2,4,5-tetrazine ring or the like), a quinoxaline ring, a pyrrole ring, a furan ring, a thiophene ring, an imidazole ring, an oxazole ring, a thiazole ring, a benzopyrrole ring, a benzofuran ring, a benzothiophene ring, a benzoimidazole ring, a benzoxazole ring, a benzothiazole ring, a naphthopyrrole ring, a naphthofuran ring, a naphthothiophene ring, a naphthimidazole ring, a naphthoxazole ring, a 3H-pyrrolidine ring, a pyrroloimidazole ring (a 5H-pyrrolo[1,2-a]imidazole ring and the like), an imidazooxazole ring (an imidazo[2,1-b]oxazole ring and the like), a thienothiazole ring (a thieno[2,3-d]thiazole ring and the like), a benzothiadiazole ring, a benzodithiophene ring (benzo[1,2-b:4,5-b′]dithiophene ring or the like), a thienothiophene ring (thieno[3,2-b]thiophene ring or the like), a thiazolothiazole ring (thiazolo[5,4-d]thiazole ring or the like), a naphthodithiophene ring (naphtho[2,3-b:6,7-b′]dithiophene ring, a naphtho[2,1-b:6,5-b′]dithiophene ring, a naphtho[1,2-b:5,6-b′]dithiophene ring, a 1,8-dithiadicyclopenta[b,g]naphthalene ring, or the like), a benzothienobenzothiophene ring, a dithieno[3,2-b:2′,3′-d]thiophene ring, and a 3,4,7,8-tetrathiadicyclopenta[a,e]pentalene ring.

In the aromatic ring which may have a substituent, a kind of the substituent that the aromatic ring may have is not particularly limited, and examples thereof include a substituent W. In a case where the aromatic ring has a substituent, the number of substituents may be 1 or more (for example, 1 to 4 or the like).

In the present specification, the term “aromatic ring group” includes, for example, a group obtained by removing one or more hydrogen atoms (for example, 1 to 5 or the like) from the aromatic ring.

In the present specification, the term “aryl group” includes, for example, a group obtained by removing one hydrogen atom from a ring corresponding to an aromatic hydrocarbon ring among the above aromatic rings.

In the present specification, the term “heteroaryl group” includes, for example, a group obtained by removing one hydrogen atom from a ring corresponding to an aromatic heterocyclic ring among the above aromatic rings.

In the present specification, the term “arylene group” includes, for example, a group obtained by removing two hydrogen atoms from a ring corresponding to an aromatic hydrocarbon ring among the above aromatic rings.

In the present specification, the term “heteroarylene group” includes, for example, a group obtained by removing two hydrogen atoms from a ring corresponding to an aromatic heterocyclic ring among the above aromatic rings.

In an aromatic ring group which may have a substituent, an aryl group which may have a substituent, a heteroaryl group which may have a substituent, an arylene group which may have a substituent, and a heteroarylene group which may have a substituent, a kind of the substituents that these groups may have is not particularly limited, and examples thereof include a substituent W. In a case where these groups each of which may have a substituent have substituents, the number of substituents may be 1 or more (for example, 1 to 4 or the like).

In the present specification, in a case where a plurality of identical symbols indicating a kind or the number of groups are present in Formula (General Formula), which indicates a chemical structure, contents of these plurality of identical symbols indicating a kind or the number of groups are independent of each other, and the contents of the identical symbols may be the same or different from each other unless otherwise specified.

In the present specification, in a case where a plurality of identical groups (alkyl groups or the like) are present in one Formula (General Formula), which indicates a chemical structure, specific contents between these plurality of identical groups are independent of each other, and the specific contents between the plurality of identical groups may be the same or different from each other, unless otherwise specified.

A bonding direction of a divalent group (for example, —CO—O—) described in the present specification is not limited unless otherwise specified. For example, in a case where Y is —COO— in the compound represented by General Formula “X-Y-Z”, the compound may be “X-O-CO-Z” or may be “X-CO-O-Z”.

In the present specification, regarding a compound that may have a geometric isomer (cis-trans isomer), a general formula or a structural formula representing the above compound may be described only in the form of either a cis isomer or a trans isomer for convenience. Even in such a case, unless otherwise specified, the form of the compound is not limited to either the cis form or the trans form, and the compound may be either the cis form or the trans form.

### Photoelectric Conversion Element

The photoelectric conversion element according to an embodiment of the present invention includes a conductive film, a photoelectric conversion film, and a transparent conductive film in this order, in which the photoelectric conversion film contains a compound represented by Formula (1) described later (hereinafter, referred to as a “specific compound”).

The mechanism capable of solving the above problems by adopting such a configuration of the photoelectric conversion element according to the embodiment of the present invention is not always clear, but the present inventors have presumed as follows.

As a main feature point, the specific compound has a donor acceptor type (DA type) structure and is a group in which A¹ (acceptor moiety) is represented by Formula (1A) or Formula (2A) described later. More specifically, the specific compound has an absorption band in a wide wavelength range of the visible light region (specifically, a wavelength range of 450 to 650 nm) based on the fact that structures of Y¹¹ and Y¹² in Formula (1A) and structures of Y²¹ and Y²² in Formula (2A) are different from each other (asymmetric structures), and A¹ (acceptor moiety) has a 5-membered aromatic ring structure (corresponding to a 5-membered aromatic ring structure having X¹¹ to X¹³ and carbon atoms respectively adjacent to X¹¹ and X¹³ in Formula (1A) as ring member atoms or a 5-membered aromatic ring structure having X²¹ to X²³ and carbon atoms respectively adjacent to X²¹ and X²³ in Formula (2A) as ring member atoms). In addition, it is considered that the specific compound is less likely to cause aggregation or the like even though the specific compound is subjected to heat treatment (annealing treatment) due to the above-described structure, and is excellent in uniformity in the film. As a result, it is presumed that the photoelectric conversion element according to the embodiment of the present invention has a small wavelength dependence of the photoelectric conversion efficiency and exhibits a high photoelectric conversion efficiency even during the heat treatment (annealing treatment).

Hereinbelow, the fact that the photoelectric conversion efficiency of the photoelectric conversion element after the heat treatment (annealing treatment) is more excellent, the variation of the photoelectric conversion efficiency of the photoelectric conversion element due to the heat treatment (annealing treatment) is more excellent, the response speed of the photoelectric conversion element is more excellent, and/or the suppression of an electric field intensity dependence of the response speed of the photoelectric conversion element is more excellent is referred to as “the effect of the present invention is more excellent”.

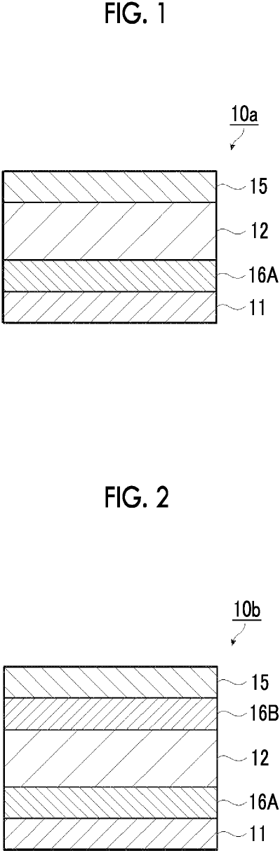

FIG. 1 is a schematic cross-sectional view of one embodiment of a photoelectric conversion element according to the embodiment of the present invention.

A photoelectric conversion element 10a illustrated in FIG. 1 has a configuration in which a conductive film (hereinafter, also referred to as a “lower electrode”) 11 functioning as a lower electrode, an electron blocking film 16A, a photoelectric conversion film 12 containing the specific compound described later, and a transparent conductive film (hereinafter, also referred to as an “upper electrode”) 15 functioning as an upper electrode are laminated in this order.

FIG. 2 illustrates a configuration example of another photoelectric conversion element. A photoelectric conversion element 10b illustrated in FIG. 2 has a configuration in which the electron blocking film 16A, the photoelectric conversion film 12, a positive hole blocking film 16B, and the upper electrode 15 are laminated on the lower electrode 11 in this order. The lamination order of the electron blocking film 16A, the photoelectric conversion film 12, and the positive hole blocking film 16B in FIGS. 1 and 2 may be appropriately changed according to the application and the characteristics.

In the photoelectric conversion element 10a (or 10b), it is preferable that light is incident on the photoelectric conversion film 12 through the upper electrode 15.

In a case where the photoelectric conversion element 10a (or 10b) is used, a voltage can be applied. In this case, it is preferable that the lower electrode 11 and the upper electrode 15 form a pair of electrodes, and a voltage is applied between the pair of electrodes.

The above-described voltage is preferably 1.0 × 10⁻⁵ to 1.0 × 10⁷ V/cm, and from the viewpoint of performance and power consumption, more preferably 1.0 × 10⁻⁴ to 1.0 × 10⁷ V/cm, and still more preferably 1.0 × 10⁻³ to 5.0 × 10⁶ V/cm.

Regarding a voltage application method, in FIGS. 1 and 2, it is preferable that the voltage is applied such that the electron blocking film 16A side is a cathode and the photoelectric conversion film 12 side is an anode. In a case where the photoelectric conversion element 10a (or 10b) is used as an optical sensor, or also in a case where the photoelectric conversion element 10a (or 10b) is incorporated in an imaging element, the voltage can be applied by the same method.

As described in detail below, the photoelectric conversion element 10a (or 10b) can be suitably applied to applications of the imaging element.

Hereinafter, the form of each layer constituting the photoelectric conversion element according to the embodiment of the present invention will be described in detail.

### Photoelectric Conversion Film

The photoelectric conversion film is a film containing a specific compound.

Hereinafter, the specific compound will be described in detail.

### Compound (Specific Compound) Represented by Formula (1)

The specific compound is the compound represented by Formula (1).

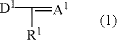

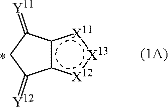

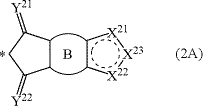

In Formula (1), A¹ represents a group represented by any one of Formula (1A) or Formula (2A).

Hereinbelow, each of the group represented by Formula (1A) and the group represented by Formula (2A) will be described.

In Formula (1A), Y¹¹ and Y¹² each independently represent an oxygen atom, a sulfur atom, ═C(CN)₂, ═C(COR¹¹)₂, ═C(SO₂R¹¹)₂, ═C(SOR¹¹)₂, =C(CN)(COR¹¹), ═C(CN)(SO₂R¹¹), ═C(CN)(SOR¹¹), ═C(COR¹¹) (SO₂R¹¹), ═C(COR¹¹)(SOR¹¹), or ═C(SO₂R¹¹)(SOR¹¹). Here, Y¹¹ and Y¹² are not the same as each other.

R¹¹ each independently represents a hydrogen atom, a hydrocarbon group which may have a substituent, or a heteroaryl group which may have a substituent.

Examples of the hydrocarbon group represented by R¹¹ include an alkyl group, an alkenyl group, an alkynyl group, and an aryl group.

An example of a suitable aspect of Y¹¹ and Y¹² includes an aspect in which one of Y¹¹ or Y¹² represents an oxygen atom or a sulfur atom.

An example of another suitable aspect of Y¹¹ and Y¹² includes an aspect in which one of Y¹¹ or Y¹² represents ═C(CN)₂.

Among these, from the viewpoint that the effect of the present invention is more excellent, it is preferable that one of Y¹¹ or Y¹² represents an oxygen atom or a sulfur atom, and the other represents ═C(CN)₂, and it is more preferable that one of Y¹¹ or Y¹² represents an oxygen atom, and the other represents ═C(CN)₂.

One of X¹¹ or X¹² represents a sulfur atom, an oxygen atom, a selenium atom, or NR¹², and the other represents a nitrogen atom or —CR^{x}═.

From the viewpoint that the effect of the present invention is more excellent, it is preferable that one of X¹¹ or X¹² represents a sulfur atom, and the other represents a nitrogen atom or —CR^{x}═, and it is more preferable that one of X¹¹ or X¹² represents a sulfur atom, and the other represents —CR^{x}═.

X¹³ represents —CR^{x}═ or a nitrogen atom.

X¹³ preferably represents —CR^{x}═ from the viewpoint that the effect of the present invention is more excellent.

R¹² represents a hydrogen atom or a substituent. R¹² is preferably a hydrogen atom.

R^{x} represents a hydrogen atom or a substituent. Among these, R^{x} is preferably a hydrogen atom, an alkyl group, an aryl group, a heteroaryl group, a halogen atom, or a cyano group, more preferably a hydrogen atom, a halogen atom, or a cyano group, still more preferably a hydrogen atom or a halogen atom, and particularly preferably a hydrogen atom or a chlorine atom. In a case where a plurality of R^{x}’s are present in Formula, R^{x}’s may be the same as or different from each other.

In a case where X¹¹ and X¹³ each represent —CR^{x}═, R^{x}’s may be bonded to each other to form a ring. In a case where X¹² and X¹³ each represent —CR^{x}═, R^{x}’s may be bonded to each other to form a ring. The above-described ring may be an aromatic ring or an alicyclic ring, but an aromatic ring is preferable, and an aromatic hydrocarbon ring is more preferable. The above-described ring is preferably a 6-membered ring. In addition, the above-described ring member atoms in the ring may include a heteroatom. Examples of the heteroatoms include a nitrogen atom, a sulfur atom, an oxygen atom, a selenium atom, a tellurium atom, a phosphorus atom, a silicon atom, and a boron atom.

The above-described ring is preferably a benzene ring which may have a substituent. The above-described ring may further have a substituent. Among these, as a substituent, a hydrogen atom, an alkyl group, an aryl group, a heteroaryl group, a halogen atom (the halogen atom is preferably a chlorine atom, a bromine atom, or an iodine atom), or a cyano group is preferable.

In Formula (1A), * represents a bonding position.

A dotted line specified in Formula (1A) represents that a ring structure having X¹¹ to X¹³ and carbon atoms respectively adjacent to X¹¹ and X¹³ in Formula (1A) as ring member atoms forms a resonance structure (in other words, the above-described ring structure is an aromatic ring).

For example, in a case where X¹¹ represents a nitrogen atom or —CR^{x}═, and X¹² represents a sulfur atom, an oxygen atom, a selenium atom, or NR¹², an example of the group represented by Formula (1A) includes a group represented by Formula (1Aa) described below. *, Y¹¹, Y¹², R^{x} X¹¹, and X¹² in Formula (1Aa) are as described above.

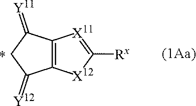

In Formula (1A), as described above, it is preferable that one of Y¹¹ or Y¹² represents an oxygen atom or a sulfur atom, and the other represents ═C(CN)₂ from the viewpoint that the effect of the present invention is more excellent.

In addition, in Formula (1A), for example, in a case where Y¹¹ represents an oxygen atom or a sulfur atom, and Y¹² represents ═C(CN)₂, it is preferable that X¹¹ represents a nitrogen atom or —CR^{x}═, and X¹² represents a sulfur atom, an oxygen atom, a selenium atom, or NR¹², it is more preferable that X¹¹ represents a nitrogen atom or —CR^{x}═, and X¹² represents a sulfur atom, and it is still more preferable X¹¹ represents —CR^{x}═, and X¹² represents a sulfur atom. In this case, it is preferable that X¹³ represents —CR^{x}═.

In addition, from the viewpoint that the effect of the present invention is more excellent, an aspect in which the group represented by Formula (1A) is a group represented by Formula (1Aa) described above where Y¹¹ represents an oxygen atom, Y¹² represents ═C(CN)₂, X¹¹ preperably represents —CR^{x}═, and X¹² preperably represents a sulfur atom (except that R^{x}’s in Formula (1Aa) are bonded to each other to form a ring).

Specifically, as the group represented by Formula (1A), a group represented by Formula (1Aa-1) is also preferable.

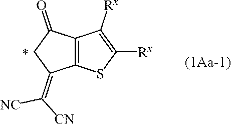

In Formula (1Aa-1), R^{x} represents a hydrogen atom or a substituent.

In Formula (2A), Y²¹ and Y²² each independently represent an oxygen atom, a sulfur atom, ═C(CN)₂, =C(COR²¹)₂, ═C(SO₂R²¹)₂, =C(SOR²¹)₂, =C(CN)(COR²¹), ═C(CN)(SO₂R²¹), =C(CN)(SOR²¹), =C(COR²¹)(SO₂R²¹), =C(COR²¹)(SOR²¹), or =C(SO₂R²¹)(SOR²¹). Here, Y²¹ and Y²² are not the same as each other.

R²¹ each independently represents a hydrogen atom, a hydrocarbon group which may have a substituent, or a heteroaryl group which may have a substituent.

Examples of the hydrocarbon group represented by R²¹ include an alkyl group, an alkenyl group, an alkynyl group, and an aryl group.

An example of a suitable aspect of Y²¹ and Y²² includes an aspect in which one of Y²¹ or Y²² represents an oxygen atom or a sulfur atom.

An example of another suitable aspect of Y²¹ and Y²² includes an aspect in which one of Y²¹ or Y²² represents ═C(CN)₂.

Among these, from the viewpoint that the effect of the present invention is more excellent, it is preferable that one of Y²¹ or Y²² represents an oxygen atom or a sulfur atom, and the other represents ═C(CN)₂, and it is more preferable that one of Y²¹ or Y²² represents an oxygen atom, and the other represents ═C(CN)₂.

B represents a 5- or 6-membered monocyclic aromatic ring which may have a substituent.

Specific examples of the 5- or 6-membered monocyclic aromatic ring represented by B include groups represented by Formula (1B) to Formula (3B) described below.

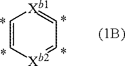

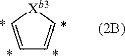

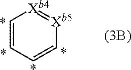

In Formula (1B), X^{b1} and X^{b2} each independently represent —CR^{z1}═ or a nitrogen atom.

R^{z1} represents a hydrogen atom or a substituent.

X^{b1} and X^{b2} are each preferably —CR^{z1}═, from the viewpoint that the effect of the present invention is more excellent. In a case where two R^{z1}′s are present in Formula (1B), the two R^{z1}′s may be the same or different from each other.

* represents a bonding position.

In Formula (2B), X^{b3} represents a sulfur atom, an oxygen atom, a selenium atom, —CR^{z2}R^{z3}—, —SiR^{z4}R^{z5}—, or —GeR^{z6}R^{z7}—.

Among these, X^{b3} is preferably a sulfur atom.

R^{z2} to R^{z7} each independently represent a hydrogen atom or a substituent.

* represents a bonding position.

In Formula (3B), X^{b4} and X^{b5} each independently represent —CR^{z8}═ or a nitrogen atom. R^{Z8} represents a hydrogen atom or a substituent.

X^{b4} and X^{b5} are each preferably —CR^{z8}═, from the viewpoint that the effect of the present invention is more excellent. In a case where two R^{z8}′s are present in Formula (3B), the two R^{z8}′s may be the same or different from each other.

* represents a bonding position.

* in the groups represented by Formula (1B) to Formula (3B) represents a bonding position as described above, and at the bonding position, a polycyclic structure is formed by bonding with two 5-membered rings adjacent to B. Examples of this polycyclic structure include structures represented by Formula (2Aa) to Formula (2Ae) described below.

One of X²¹ or X²² represents a sulfur atom, an oxygen atom, a selenium atom, or NR²², and the other represents a nitrogen atom or —CR^{y}═.

From the viewpoint that the effect of the present invention is more excellent, it is preferable that one of X²¹ or X²² represents a sulfur atom, and the other represents a nitrogen atom or -CR^{Y}=, and it is more preferable that one of X²¹ and X²² represents a sulfur atom, and the other represents -CR^{Y}=.

X²³ represents —CR^{y}═ or a nitrogen atom.

X²³ preferably represents —CR^{y}═ from the viewpoint that the effect of the present invention is more excellent.

R²² represents a hydrogen atom or a substituent. R²² is preferably a hydrogen atom.

R^{y} represents a hydrogen atom or a substituent. Among these, R^{y} is preferably a hydrogen atom, an alkyl group, an aryl group, a heteroaryl group, a halogen atom, or a cyano group, more preferably a hydrogen atom, a halogen atom, or a cyano group, still more preferably a hydrogen atom or a halogen atom, and particularly preferably a hydrogen atom or a chlorine atom. In a case where a plurality of R^{y}’s are present in Formula, R^{y}’s may be the same as or different from each other.

In a case where X²¹ and X²³ each represent —CR^{y}═, R^{y}’s may be bonded to each other to form a ring. In a case where X²² and X²³ each represent -CR^{Y}=, R^{y}’s may be bonded to each other to form a ring. The above-described ring may be an aromatic ring or an alicyclic ring, but an aromatic ring is preferable, and an aromatic hydrocarbon ring is more preferable. The above-described ring is preferably a 6-membered ring. In addition, the above-described ring member atoms in the ring may include a heteroatom.

Examples of the heteroatoms include a nitrogen atom, a sulfur atom, an oxygen atom, a selenium atom, a tellurium atom, a phosphorus atom, a silicon atom, and a boron atom.

The above-described ring is preferably a benzene ring which may have a substituent.

The above-described ring may further have a substituent. Among these, as a substituent, a hydrogen atom, an alkyl group, an aryl group, a heteroaryl group, a halogen atom (the halogen atom is preferably a chlorine atom, a bromine atom, or an iodine atom), or a cyano group is preferable.

A dotted line specified in Formula (2A) represents that a ring structure having X²¹ to X²³ and carbon atoms respectively adjacent to X²¹ and X²³ in Formula (2A) as ring member atoms forms a resonance structure (in other words, the above-described ring structure is an aromatic ring).

For example, in a case where X²¹ represents a nitrogen atom or -CR^{Y}=, and X²² represents a sulfur atom, an oxygen atom, a selenium atom, or NR²², examples of the group represented by Formula (2A) include groups represented by Formula (2Aa) to Formula (2Ae) described below. In addition, *, Y²¹, Y²², X^{b1} to X^{b5}, R^{y}, X²¹, and X²² in Formula (2Aa) to Formula (2Ae) are as described above.

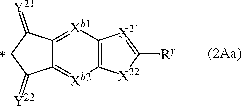

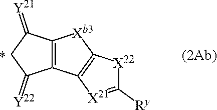

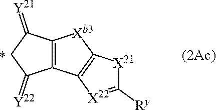

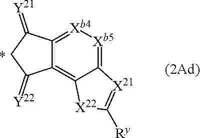

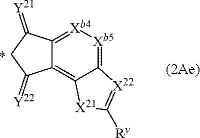

* represents a bonding position.

In Formula (1), A¹ is preferably a group represented by Formula (1A) from the viewpoint that the effect of the present invention is more likely to be excellent.

From the viewpoint that the effect of the present invention is more excellent, the group represented by Formula (1A) and the group represented by Formula (2A) preferably do not contain a fluorine atom. In other words, it is preferable that A¹ in Formula (1) does not have a fluorine atom.

In Formula (1), D¹ represents a substituent having a nitrogen atom and an aromatic ring.

The substituent having an aromatic ring means a substituent having an aromatic ring in a part or all of the substituent. In addition, the above-described substituent having an aromatic ring further contains a nitrogen atom. The nitrogen atom may be contained as a ring member atom of an aromatic ring, or may be contained at a position other than the ring member atom of the aromatic ring.

From the viewpoint that the effect of the present invention is more excellent, D¹ is preferably a substituent having an aromatic ring containing a nitrogen atom as a ring member atom or a substituent having a fused ring formed by an aromatic ring being fused with another ring containing a nitrogen atom, and more preferably a substituent having a fused ring formed by an aromatic ring being fused with another ring containing a nitrogen atom. In the fused ring, the aromatic ring may be a nitrogen-containing aromatic ring.

The aromatic ring contained in D¹ may be any one of a monocyclic ring or a polycyclic ring.

The number of ring members of the aromatic ring contained in D¹ is not particularly limited, and is preferably 5 to 40, and more preferably 5 to 30, for example.

Examples of the aromatic ring contained in D¹ include an aromatic hydrocarbon ring and an aromatic heterocyclic ring.

The aromatic hydrocarbon ring is not particularly limited, and examples thereof include a benzene ring, a naphthalene ring, an anthracene ring, a phenanthrene ring, and other rings.

The aromatic heterocyclic ring is not particularly limited, and examples thereof include a thiophene ring, a furan ring, a pyran ring, a thiazole ring, a pyrrole ring, a pyridine ring, a pyrazine ring, a pyrimidine ring, a pyridazine ring, an oxazole ring, a selenophene ring, an imidazole ring, a quinoxaline ring, a benzothiazole ring, and other rings.

In addition, a substituent having an aromatic ring represented by D¹ may further have another ring, or the aromatic ring and the other ring may be fused to form a fused ring.

Examples of the other ring include an alicyclic ring such as a cycloalkane ring, and an alicyclic ring containing a nitrogen atom as a ring member atom, such as a piperidine ring, a piperazine ring, and an imidazolidine ring. The alicyclic ring containing a nitrogen atom as a ring member atom is preferably a 5- to 6-membered ring, and preferably contains 1 to 3 nitrogen atoms.

From the viewpoint that the effect of the present invention is more excellent, among these, the substituent having an aromatic ring represented by D¹ preferably has a substituent having a fused ring formed by an aromatic ring and an alicyclic ring that contains a nitrogen atom as a ring member atom being fused. In the fused ring, the aromatic ring may be a nitrogen-containing aromatic ring.

D¹ is preferably a group represented by any one of Formula (1D) described below and Formula (2D) described below.

Hereinbelow, the group represented by (1D) will be described.

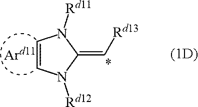

In Formula (1D), * represents a bonding position.

Ar^{d11} represents an aromatic ring containing two or more carbon atoms, which may have a substituent. That is, Ar^{d11} represents an aromatic ring containing at least two carbon atoms (intended to two carbon atoms specified in Formula (1D)), which may have a substituent.

Ar^{d11} is preferably an aromatic heterocyclic ring, and more preferably a quinoxaline ring or a pyrazine ring.

Examples of the substituent having the aromatic ring represented by Ar^{d11} include a group exemplified by the substituent W, and an alkyl group which may have a substituent, a halogen atom, or a cyano group is preferable, and an alkyl group which may have a substituent or a chlorine atom is more preferable.

R^{d11} and R^{d12} each independently represent an aromatic ring group or -C(R^{L11})(R^{L12})(R^{L13}), which may have a substituent.

Examples of the aromatic ring group include an aryl group and a heteroaryl group, and among these, an aryl group is preferable, a phenyl group, a naphthyl group, or a fluorenyl group is more preferable, and a phenyl group or a naphthyl group is still more preferable.

In addition, examples of the substituent that the aromatic ring group may have include a group exemplified by the substituent W, and among these, an alkyl group (preferably having 1 to 3 carbon atoms) or a chlorine atom is preferable, and an alkyl group (preferably having 1 to 3 carbon atoms) is more preferable.

In particular, in a case where the aromatic ring group is a phenyl group, the phenyl group preferably has a substituent (in other words, the aromatic ring group is preferably a phenyl group having a substituent). The number of the substituent is preferably 1 to 5 and more preferably 1 to 3.

R^{L11} to R^{L13} each independently represent a hydrogen atom, an alkyl group which may have a substituent, or an aromatic ring group which may have a substituent.

The alkyl group may be any one of linear, branched, or cyclic.

Examples of the aromatic ring group include an aryl group and a heteroaryl group, and among these, an aryl group is preferable, a phenyl group, a naphthyl group, or a fluorenyl group is more preferable, and a phenyl group or a naphthyl group is still more preferable.

Examples of the substituent that the alkyl group and the aromatic ring group may have include a group exemplified by the substituent W, and among these, an alkyl group (preferably having 1 to 3 carbon atoms) or a chlorine atom is preferable, and an alkyl group (preferably having 1 to 3 carbon atoms) is more preferable.

R^{L11} to R^{L13} may be bonded to each other to form a ring. That is, an alkyl group which may have a substituent and an aromatic ring group which may have a substituent (preferably, an aryl group which may have a substituent and a heteroaryl group which may have a substituent), which are represented by R^{L11} to R^{L13}, may be bonded to each other to form a ring.

Hereinbelow, aspects in which R^{L11} to R^{L13} are bonded to each other to form a ring will be described.

For example, the alkyl groups which may have a substituent may be bonded to each other to form a ring. A substituent in the aryl group which may have a substituent and the alkyl group which may have a substituent may be bonded to each other to form a ring. A substituent in the heteroaryl group which may have a substituent and the alkyl group which may have a substituent may be bonded to each other to form a ring. A substituent in the aryl group which may have a substituent and a substituent in another aryl group which may have a substituent may be bonded to each other to form a ring. A substituent in the aryl group which may have a substituent and a substituent in the heteroaryl group which may have a substituent may be bonded to each other to form a ring. A substituent in the heteroaryl group which may have a substituent and a substituent in another heteroaryl group which may have a substituent may be bonded to each other to form a ring.

A substituent in the ring formed as described above, and another alkyl group which may have a substituent, a substituent in another aryl group which may have a substituent, or a substituent in another heteroaryl group which may have a substituent may be bonded to form a ring.

As described above, a group may be formed by bonding the substituent and the substituent (for example, the substituent in the aryl group which may have a substituent and the substituent in the heteroaryl group which may have a substituent) to form a single bond.

In a case where the alkyl group which may have a substituent and the aromatic ring group which may have a substituent (preferably the aryl group which may have a substituent or the heteroaryl group which may have a substituent), which are represented by R^{L11} to R^{L13}, are bonded to each other to form a ring, -C(R^{L11})(R^{L12})(R^{L13}) is preferably a group other than the aryl group and the heteroaryl group.

In a case where R^{L11} to R^{L13} are bonded to each other to form a ring, the ring is preferably a monocyclic or polycyclic cycloalkane ring from the viewpoint that the effect of the present invention is more excellent.

Hereinbelow, aspects in which R^{L11} to R^{L13} are bonded to each other to form a monocyclic or polycyclic cycloalkane ring will be described.

In a case where R^{L11} to R^{L13} are bonded to each other to form a ring, an alkyl group represented by R^{L11} and an alkyl group represented by R^{L12} may be bonded to each other to form a ring, or a substituent contained in a ring (for example, a monocyclic cycloalkane ring or the like) formed by bonding an alkyl group represented by R^{L11} and an alkyl group represented by R^{L12} and an alkyl group represented by R^{L13} may be bonded to each other to form a polycyclic ring (for example, a polycyclic cycloalkane ring or the like). That is, -C(R^{L11})(R^{L12})(R^{L13}) may be a cycloalkyl group (preferably a cyclohexyl group) which may have a substituent. The number of membered rings of the cycloalkyl group is preferably 3 to 12, more preferably 3 to 8, and still more preferably 3 to 6.

The cycloalkyl group may be a monocyclic ring (for example, a cyclohexyl group or the like) or a polycyclic ring (1-adamantyl group or the like).

The cycloalkyl group may have a substituent. Examples of the substituent include a group exemplified as the substituent W, and among these, an alkyl group (preferably having 1 to 3 carbon atoms) is preferable. Substituents contained in the cycloalkyl group may be bonded to each other to form a ring, and the ring formed by bonding the substituents to each other may be a ring other than a cycloalkane ring.

Among these, R^{d11} and R^{d12} are each independently preferably an aromatic ring group which may have a substituent or an alkyl group which may have a substituent (examples of the alkyl group include alkyl groups that are linear and branched, and a cycloalkyl group), and more preferably a group represented by Formula (X) or an alkyl group which may have a substituent (examples of the alkyl group include alkyl groups that are linear and branched, and a cycloalkyl group).

The group represented by Formula (X) is preferably a group represented by Formula (Z) described later, and more preferably a group represented by Formula (ZB) described later.

R^{d11} and R^{d12} preferably have structures different from each other, from the viewpoint that the effect of the present invention is more excellent. Among these, one of R^{d11} or R^{d12} preferably represents a group represented by Formula (X) (among these, a group represented by Formula (ZB) described later is preferable), and the other preferably represents an alkyl group which may have a substituent (examples of the alkyl group include alkyl groups that are linear and branched, and a cycloalkyl group).

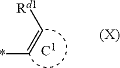

In Formula (X), * represents a bonding position.

C¹ represents a monocyclic aromatic ring containing at least two carbon atoms (which means two carbon atoms specified in Formula (X)) which may have a substituent, in addition to R^{d1}

Examples of the monocyclic aromatic ring include a monocyclic aromatic hydrocarbon ring and a monocyclic aromatic heterocyclic ring. An example of the aromatic hydrocarbon ring includes a benzene ring. Examples of the aromatic heterocyclic ring include a pyrrole ring, a furan ring, a thiophene ring, an imidazole ring, a thiazole ring, a pyridine ring, and an oxazole ring.

As the monocyclic aromatic ring, an aromatic hydrocarbon ring is preferable and a benzene ring is more preferable, from the viewpoint that the effect of the present invention is more excellent.

R^{d1} represents an alkyl group, a silyl group, an alkoxy group, an alkylthio group, a cyano group, a halogen atom, an aryl group, a heteroaryl group, an alkenyl group, or an alkynyl group, is preferably an alkyl group, a silyl group, an alkoxy group, an alkylthio group, a cyano group, a halogen atom, an aryl group, a heteroaryl group, an alkenyl group, or an alkynyl group, and more preferably a halogen atom or an alkyl group. These groups each may further have a substituent as much as possible.

Examples of the halogen atom represented by R^{d1} include a fluorine atom, an iodine atom, a bromine atom, and a chlorine atom, a fluorine atom or a chlorine atom is preferable, and a chlorine atom is more preferable.

Substituents which are contained in R^{d1} and C¹ may be bonded to each other to form a non-aromatic ring.

The aromatic ring of C¹ is directly bonded to the nitrogen atom specified in Formula (1D).

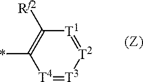

In Formula (Z), T¹ to T⁴ each independently represent —CR^{e12}═ or a nitrogen atom (═N—). R^{e12} represents a hydrogen atom or a substituent.

It is preferable that at least one of T¹, T², T³, or T⁴ represents —CR^{e12}═ and at least one of R^{e12}′s represents a substituent, and it is more preferable that at least T⁴ represents —CR^{e12}═, and R^{e12} in T⁴ represents an alkyl group, an aryl group, or a heteroaryl group.

Examples of the substituent represented by R^{e12} include a group exemplified as the substituent W, and among these, an alkyl group, an aryl group, a heteroaryl group, a silyl group, a halogen atom, or a cyano group is preferable, and an alkyl group, an aryl group, a heteroaryl group, a silyl group, a halogen atom, or a cyano group is more preferable. These groups may further have a substituent. Examples of the halogen atom represented by R^{e12} include a fluorine atom, an iodine atom, a bromine atom, and a chlorine atom, a fluorine atom or a chlorine atom is preferable, and a chlorine atom is more preferable.

In addition, in a case where a plurality of R^{e12}′s exist in Formula (Z), R^{e12}′s may be the same or different from each other.

R^{f2} in Formula (Z) has the same definition as R^{d1} in Formula (X), and the suitable embodiments thereof are also the same.

R^{f2} and R^{e12} in T¹ may be bonded to each other to form a non-aromatic ring.

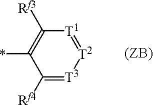

In Formula (ZB), T¹ to T³ each independently represent —CR^{e12}═ or a nitrogen atom. R^{e12} represents a hydrogen atom or a substituent.

R^{e12} in Formula (ZB) has the same definition as R^{e12} in Formula (Z), and the suitable embodiments thereof are also the same.

In Formula (ZB), R^{f3} and R^{f4} each independently represent an alkyl group, an aryl group, or a heteroaryl group. These groups each may further have a substituent as much as possible.

* represents a bonding position.

In Formula (1D), R^{d13} represents a hydrogen atom or a substituent.

R^{d13} is preferably a hydrogen atom.

The group represented by Formula (1D) described above is preferably a group represented by Formula (3D) described later, more preferably a group represented by Formula (4D) described later, and still more preferably a group represented by Formula (5D) described later, from the viewpoint that the effect of the present invention is more excellent.

Hereinafter, each of Formula (3D) to Formula (5D) will be described.

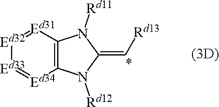

In Formula (3D), * and R^{d11} to R^{d13} have the same definition as * and R^{d11} to R^{d13} in Formula (1D) described above, and the suitable embodiments thereof are also the same.

E^{d31} to E^{d34} each independently represent a nitrogen atom or -CR^{E31}=. R^{E31} represents a hydrogen atom or a substituent.

In a case where a plurality of R^{E31}′s exist, R^{E31}′s may be bonded to each other to form a ring.

At least two of E^{d31} to E^{d34} are preferably nitrogen atoms, at least E^{d31} and E^{d34} are more preferably nitrogen atoms, and only E^{d31} and E^{d34} are still more preferably nitrogen atoms.

The ring formed by bonding R^{E31}′s to each other is preferably an aromatic ring, and more preferably a benzene ring or a pyridine ring. The ring formed by bonding R^{E31}′s to each other may further have a substituent.

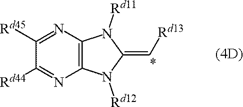

In Formula (4D), * and R^{d11} to R^{d13} have the same definition as * and R^{d11} to R^{d13} in Formula (1D) described above, and the suitable embodiments thereof are also the same.

R^{d44} and R^{d45} each independently represent a hydrogen atom or a substituent.

R^{d44} and R^{d45} may be bonded to each other to form a ring. The ring formed by bonding R^{d44} and R^{d45} to each other is preferably an aromatic ring, more preferably a benzene ring or a pyridine ring, and still more preferably a benzene ring. The ring formed by bonding R^{d44} and R^{d45} to each other may further have a substituent.

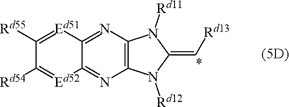

In Formula (5D), * and R^{d11} to R^{d13} have the same definition as * and R^{d11} to R^{d13} in Formula (1D) described above, and the suitable embodiments thereof are also the same.

E^{d51} and E^{d52} each independently represent a nitrogen atom or -CR^{E51}=.

R^{E51} represents a hydrogen atom or a substituent.

R^{E51} is preferably a hydrogen atom.

R^{d54} and R^{d55} each independently represent a hydrogen atom or a substituent.

Examples of the substituents represented by R^{d54} and R^{d55} include a group exemplified as the substituent W, and among these, a halogen atom or a cyano group is preferable, a fluorine atom, a chlorine atom, or a cyano group is more preferable, and a chlorine atom is particularly preferable.

R^{d54} and R^{d55} may be bonded to each other to form a ring. The ring formed by bonding R^{d54} and R^{d55} to each other is preferably an aromatic ring, and more preferably a benzene ring or a pyridine ring. The ring formed by bonding R^{d54} and R^{d55} to each other may further have a substituent.

Hereinbelow, the group represented by (2D) will be described.

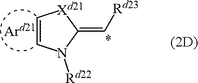

In Formula (2D), * represents a bonding position.

Ar^{d21} represents an aromatic ring containing two or more carbon atoms, which may have a substituent. That is, Ar^{d21} represents an aromatic ring containing at least two carbon atoms (intended to two carbon atoms specified in Formula (2D)), which may have a substituent.

Ar^{d21} has the same definition as Ar^{d21} in Formula (1D), and the suitable embodiments thereof are also the same. Among these, Ar^{d21} is preferably a benzene ring or a naphthalene ring, which may have a substituent.

R^{d22} represents an aromatic ring group which may have a substituent or -C(R^{L21})(R^{L22})(R^{L23}). R^{L21} to R^{L23} each independently represent a hydrogen atom, an alkyl group which may have a substituent, or an aromatic ring group which may have a substituent. R^{L21} to R^{L23} may be bonded to each other to form a ring.

R^{d22} has the same definition as R^{d12} in Formula (1D), and the suitable embodiments thereof are also the same. R^{L21} to R^{L23} have the same definition as R^{L11} to R^{L13} in R^{d12} in Formula (1D), and the suitable embodiments thereof are also the same.

X^{d21} represents a sulfur atom, an oxygen atom, or -C(R^{L24})(R^{L25})-.

R^{L24} and R^{L25} each independently represent a hydrogen atom, an alkyl group which may have a substituent, or an aromatic ring group which may have a substituent.

The alkyl group may be any one of linear, branched, or cyclic.

Examples of the aromatic ring group include an aryl group and a heteroaryl group, and among these, an aryl group is preferable, a phenyl group, a naphthyl group, or a fluorenyl group is more preferable, and a phenyl group or a naphthyl group is still more preferable.

Examples of the substituent that the alkyl group and the aromatic ring group may have include a group exemplified by the substituent W, and among these, an alkyl group (preferably having 1 to 3 carbon atoms) or a chlorine atom is preferable, and an alkyl group (preferably having 1 to 3 carbon atoms) is more preferable.

R^{L24} and R^{L25} may be bonded to each other to form a ring.

As the ring that is formed by bonding R^{L24} and R^{L25} to each other, a monocyclic or polycyclic cycloalkane ring is preferable. The number of membered rings of the cycloalkyl ring is preferably 3 to 12, more preferably 3 to 8, and still more preferably 3 to 6.

The cycloalkyl ring may have a substituent. Examples of the substituent include a group exemplified as the substituent W, and among these, an alkyl group (preferably having 1 to 3 carbon atoms) is preferable.

R^{d23} represents a hydrogen atom or a substituent.

R^{d23} is preferably a hydrogen atom.

The group represented by Formula (2D) is preferably a group represented by Formula (6D) described later from the viewpoint that the effect of the present invention is more excellent.

Hereinafter, Formula (6D) will be described.

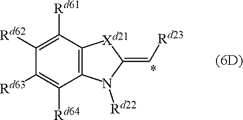

*, X^{d21}, R^{d22}, and R^{d23} in Formula (6D) have the same definition as *, X^{d21}, R^{d22}, and R^{d23} in Formula (2D), and the suitable embodiments thereof are also the same.

R^{d61} to R^{d64} each independently represent a hydrogen atom or a substituent.

Examples of the substituents represented by R^{d61} to R^{d64} include a group exemplified as the substituent W, and a halogen atom or a cyano group is preferable, a fluorine atom, a chlorine atom, or a cyano group is more preferable, and a chlorine atom is particularly preferable.

In addition, R^{d61} to R^{d64} may be bonded to each other to form a ring (preferably, R^{d61} and R^{d62}, R^{d62} and R^{d63}, and/or R^{d63} and R^{d64} are bonded to each other to form a ring). The ring formed by bonding R^{d61} to R^{d64} to each other is preferably an aromatic ring, and more preferably a benzene ring or a pyridine ring. The ring formed by bonding R^{d61} to R^{d64} to each other may further have a substituent.

Formula (6D) preferably satisfies at least one or more following requirements from the viewpoint that the effect of the present invention is more excellent.

Requirement X1: R^{d22} represents an aromatic ring group which may have a substituent.

Requirement X2: X^{d21} represents -C(R^{L24})(R^{L25})-, and R^{L24} and R^{L25} are bonded to each other to form a ring.

Requirement X3: any one or more of R^{d61} and R^{d62}, R^{d62} and R^{d63}, and R^{d63} and R^{d64} are bonded to each other to form a ring.

From the viewpoint that the effect of the present invention is more excellent, the group represented by Formula (1D) and the group represented by Formula (2D) preferably do not contain a fluorine atom. In other words, it is preferable that D¹ in Formula (1) does not have a fluorine atom.

In Formula (1), R¹ represents a hydrogen atom or a substituent.

R¹ is preferably a hydrogen atom.

Hereinbelow, specific examples of the specific compound will be described, but the specific compound in the present invention is not limited thereto.

In addition, “R” in structural formulae of compounds illustrated below represents any one selected from the group illustrated in <<R Group>> described later. * represented by a group represented by <<R Group>> represents a bonding position. In addition, “R” in the structural formulae of the compounds described below corresponds to the groups represented by A¹ in Formula (1) described above.

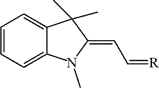

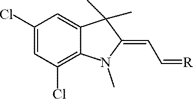

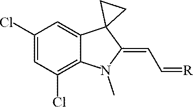

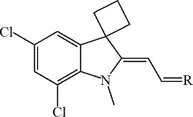

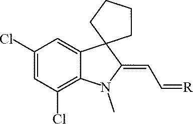

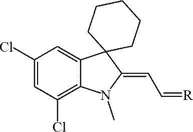

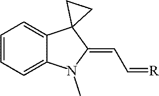

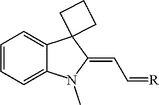

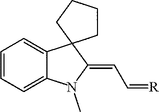

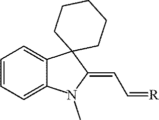

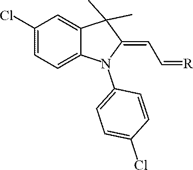

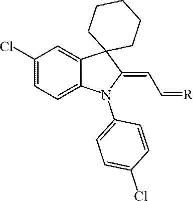

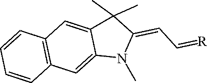

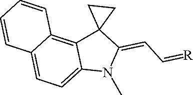

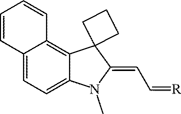

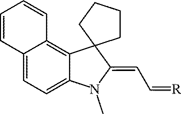

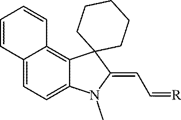

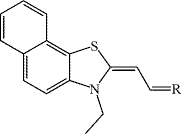

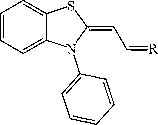

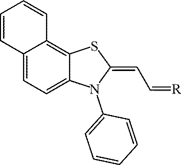

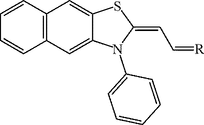

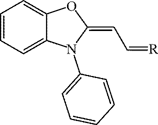

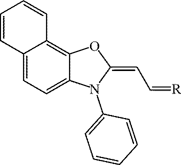

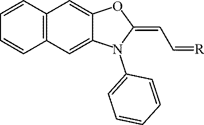

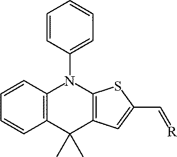

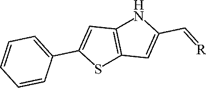

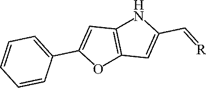

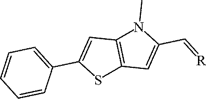

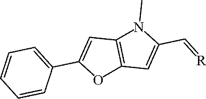

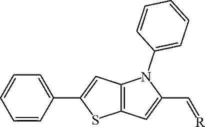

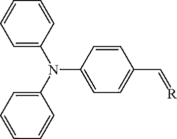

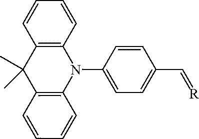

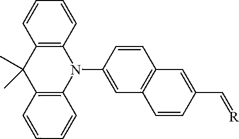

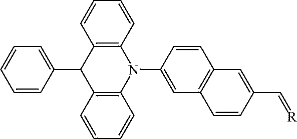

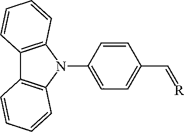

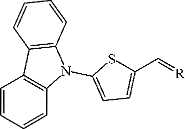

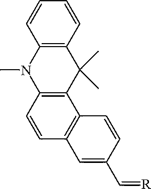

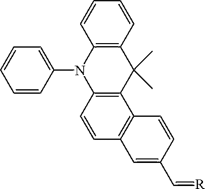

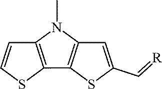

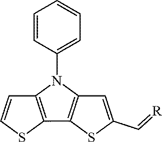

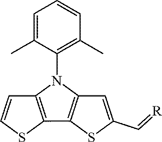

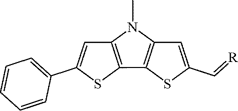

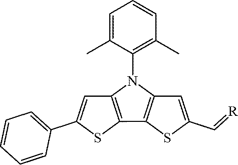

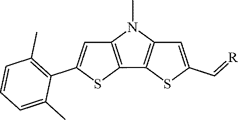

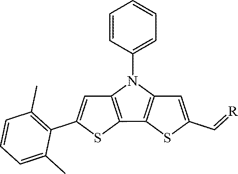

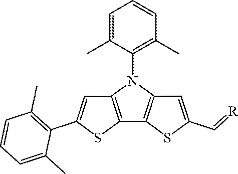

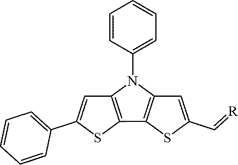

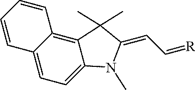

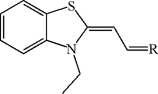

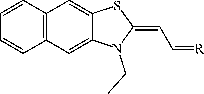

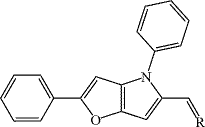

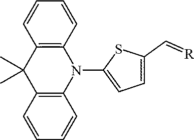

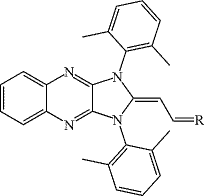

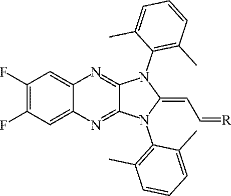

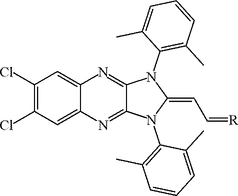

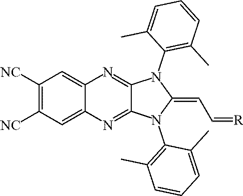

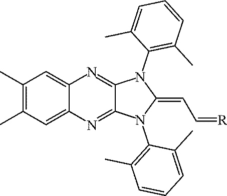

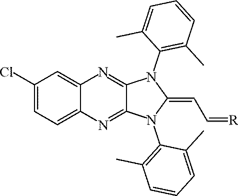

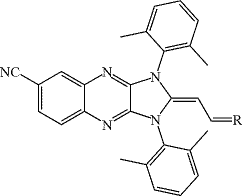

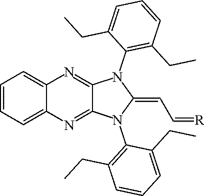

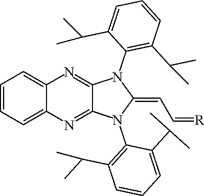

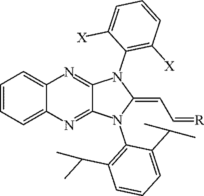

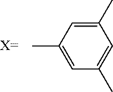

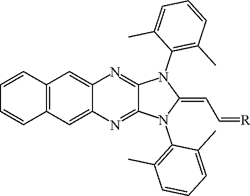

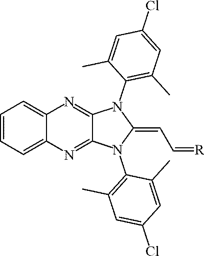

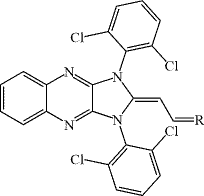

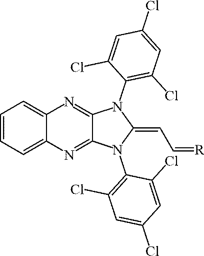

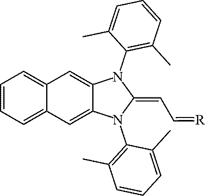

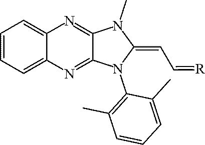

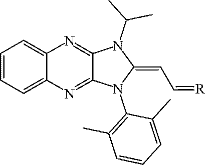

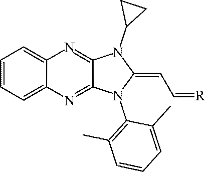

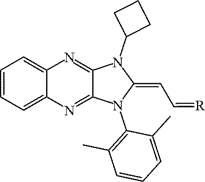

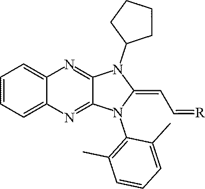

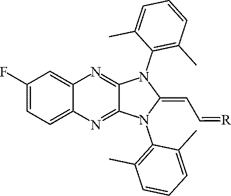

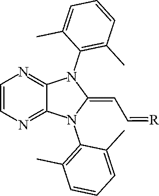

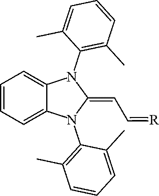

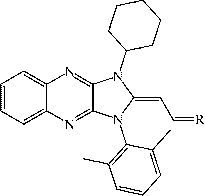

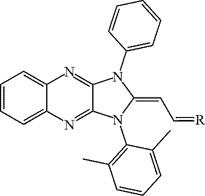

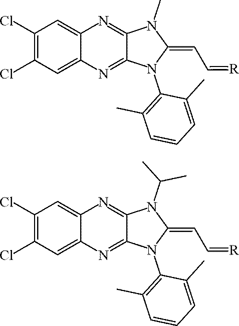

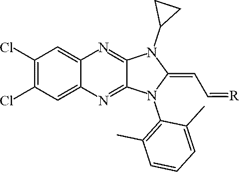

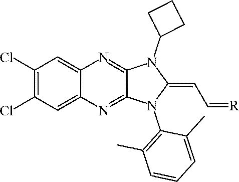

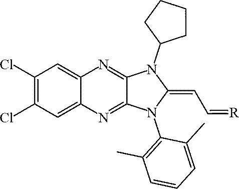

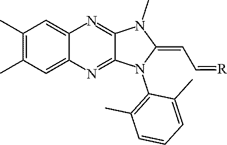

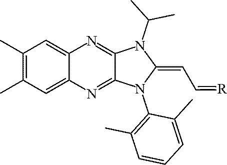

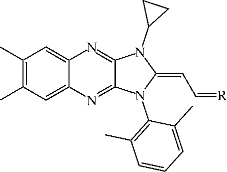

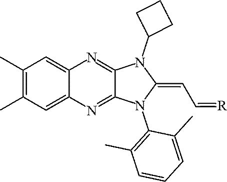

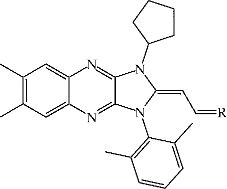

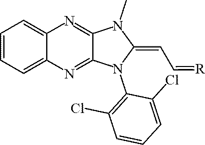

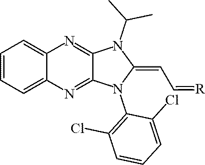

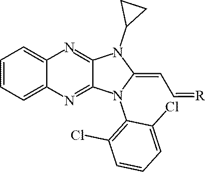

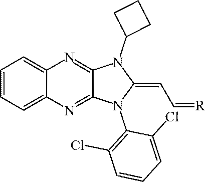

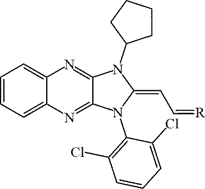

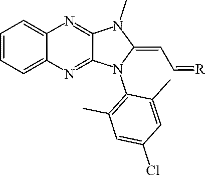

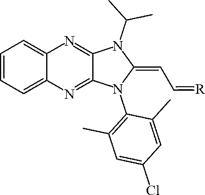

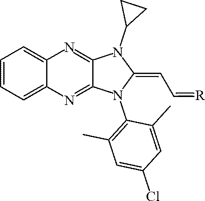

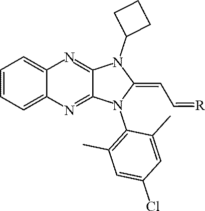

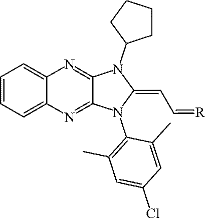

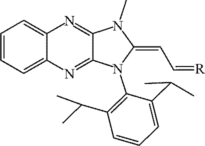

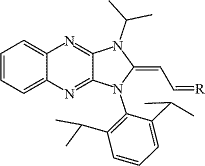

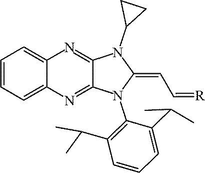

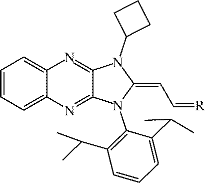

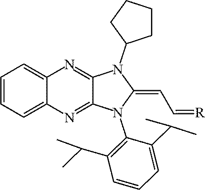

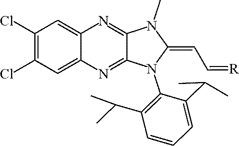

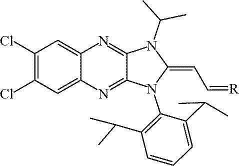

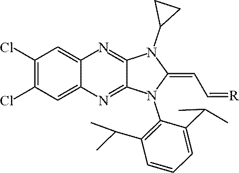

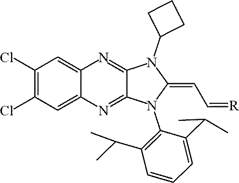

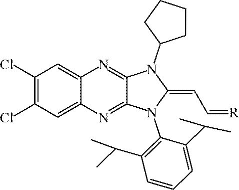

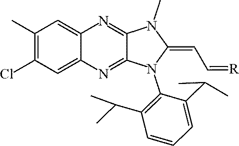

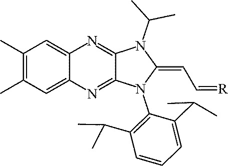

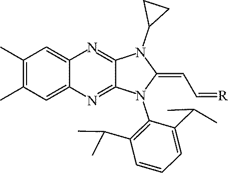

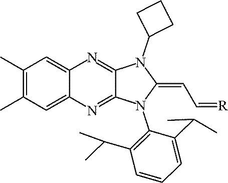

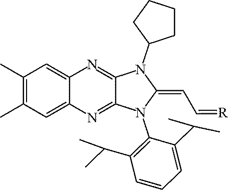

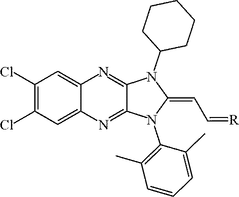

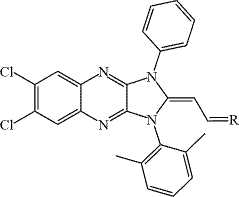

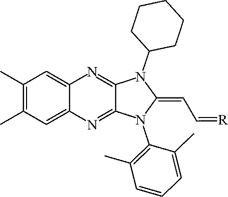

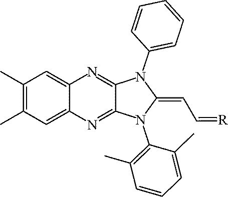

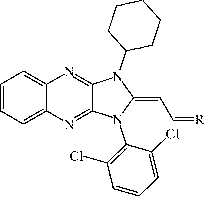

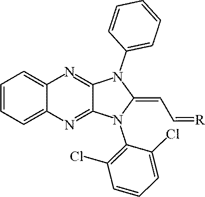

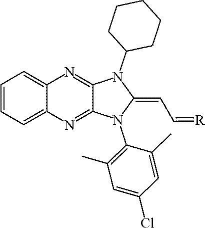

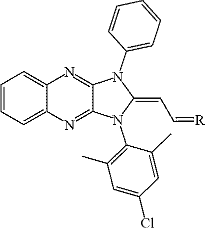

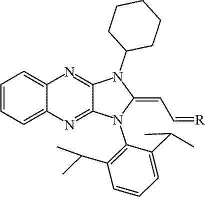

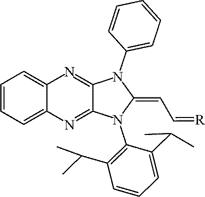

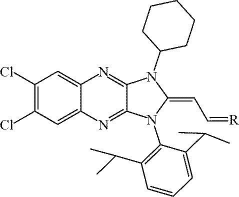

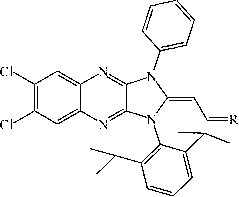

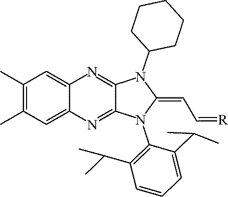

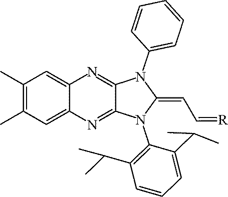

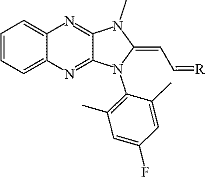

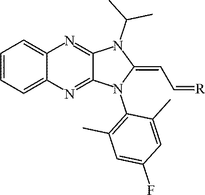

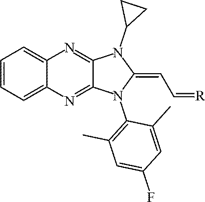

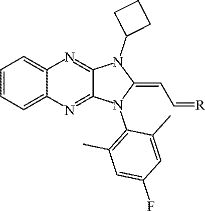

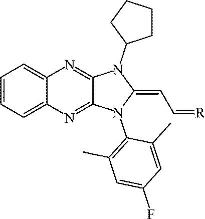

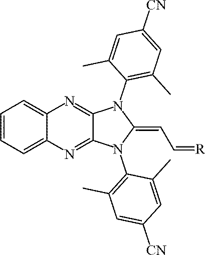

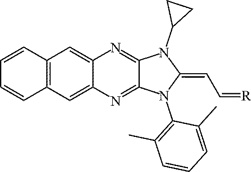

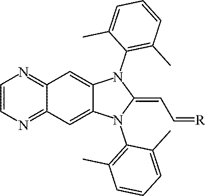

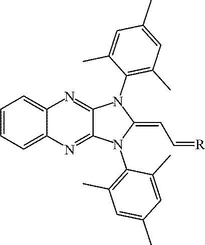

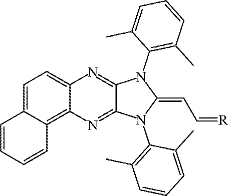

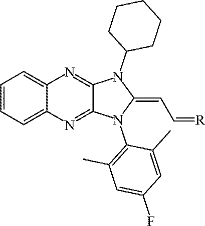

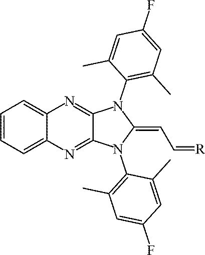

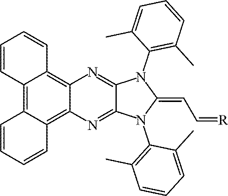

R Group

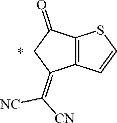

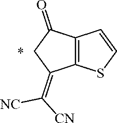

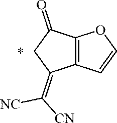

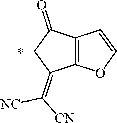

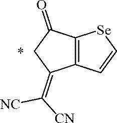

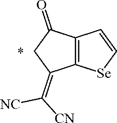

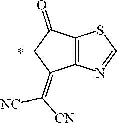

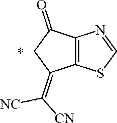

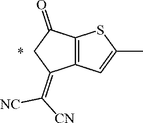

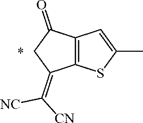

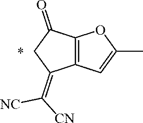

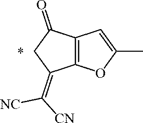

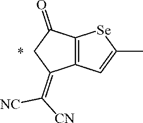

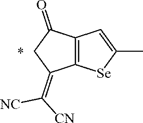

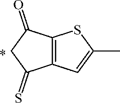

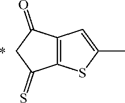

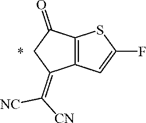

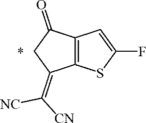

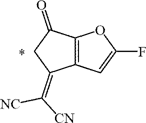

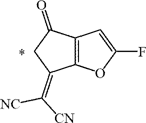

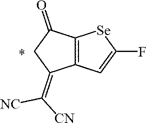

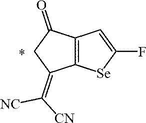

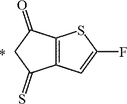

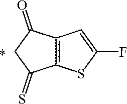

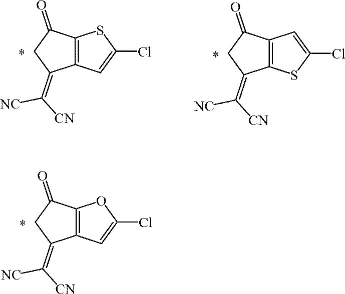

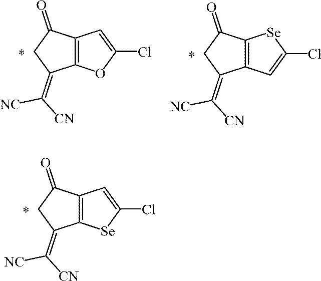

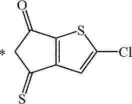

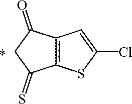

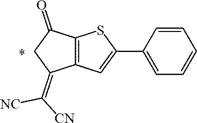

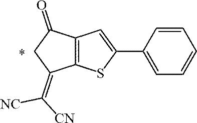

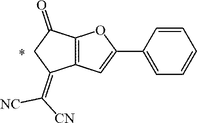

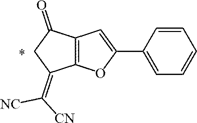

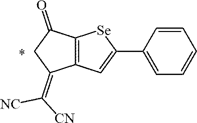

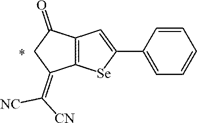

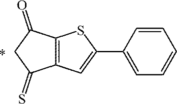

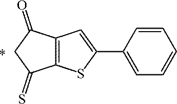

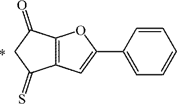

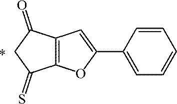

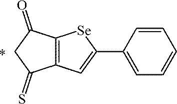

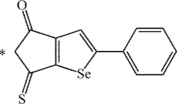

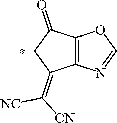

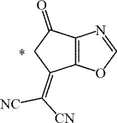

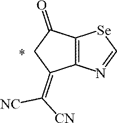

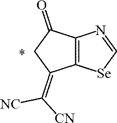

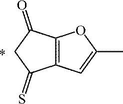

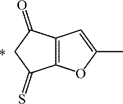

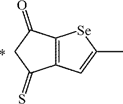

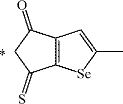

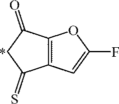

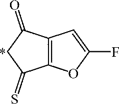

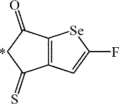

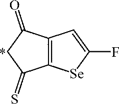

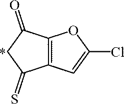

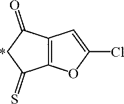

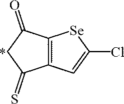

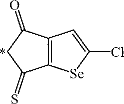

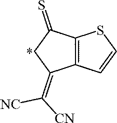

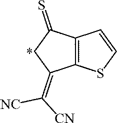

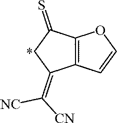

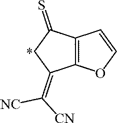

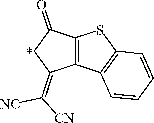

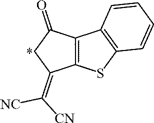

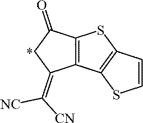

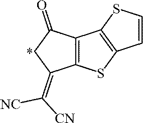

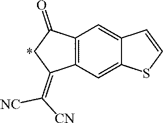

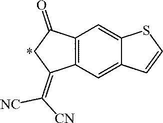

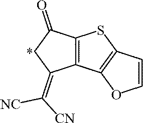

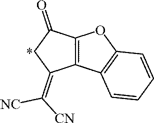

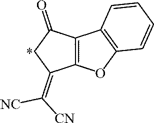

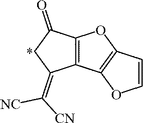

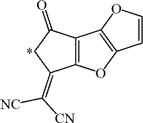

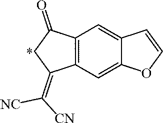

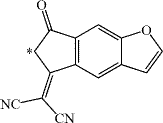

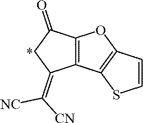

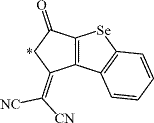

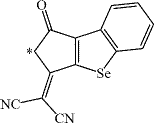

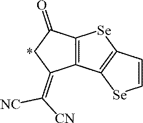

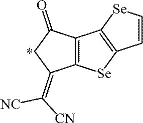

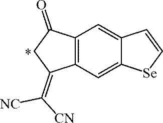

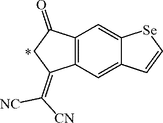

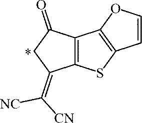

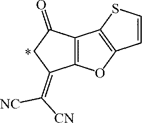

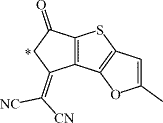

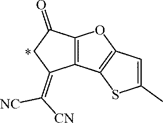

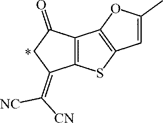

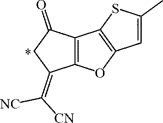

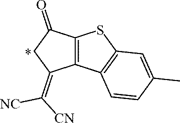

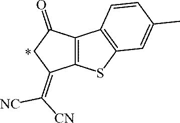

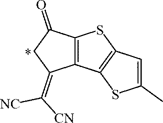

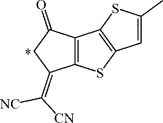

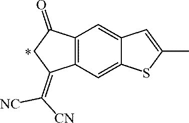

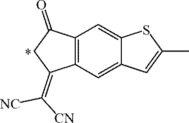

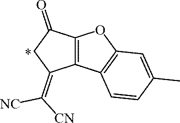

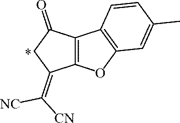

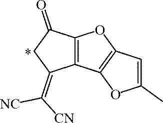

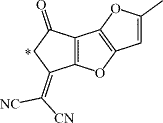

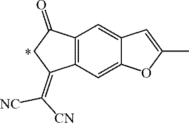

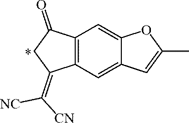

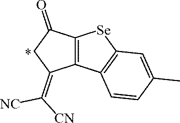

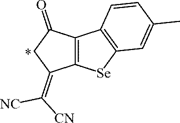

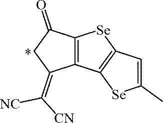

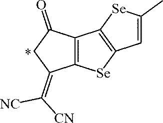

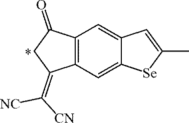

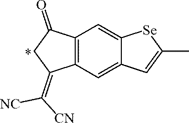

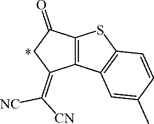

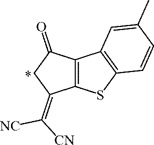

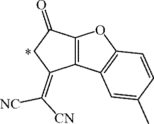

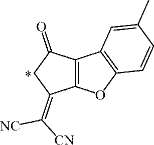

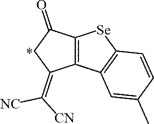

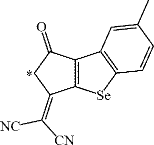

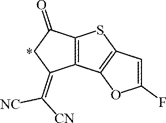

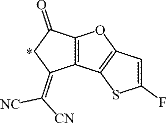

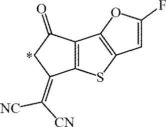

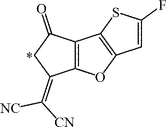

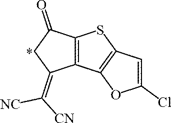

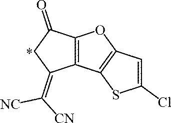

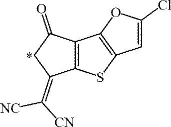

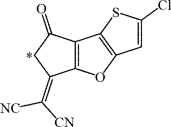

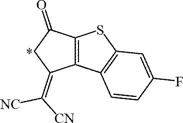

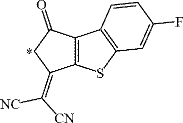

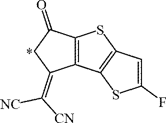

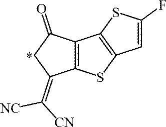

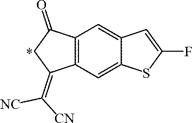

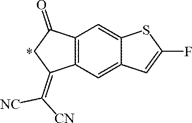

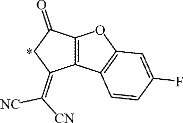

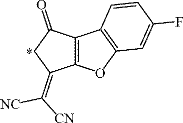

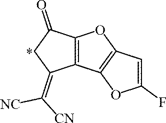

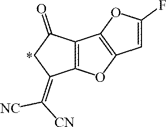

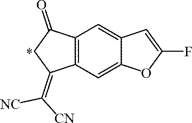

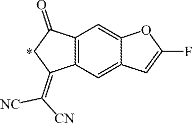

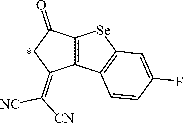

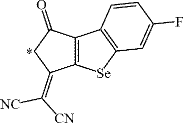

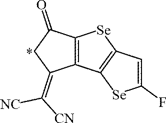

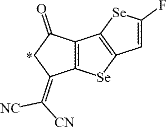

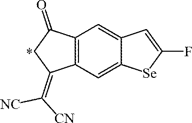

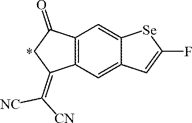

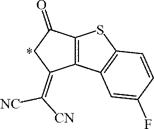

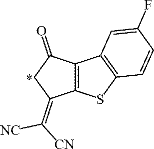

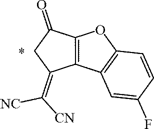

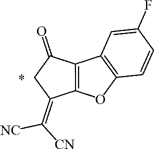

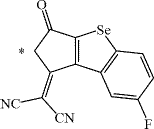

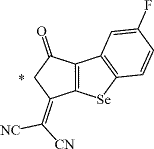

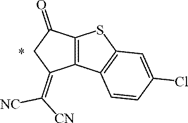

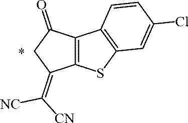

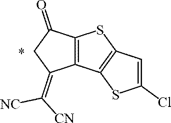

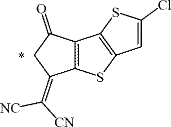

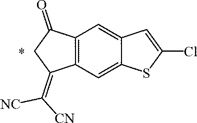

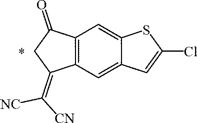

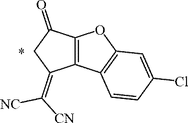

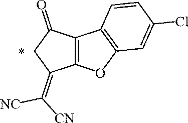

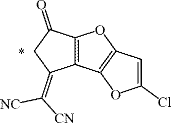

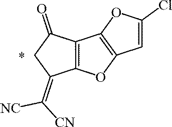

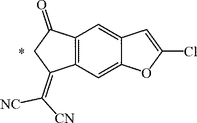

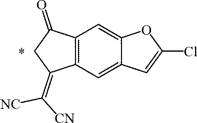

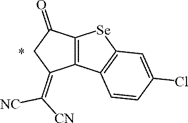

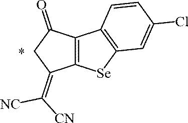

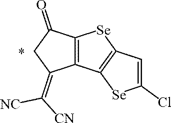

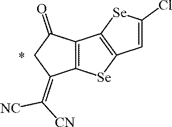

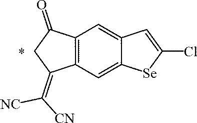

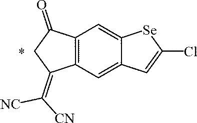

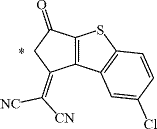

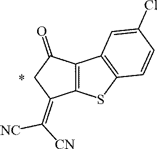

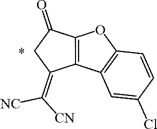

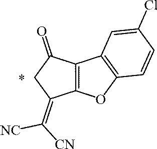

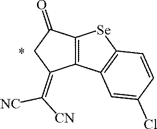

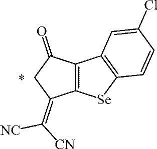

A molecular weight of the specific compound is preferably 350 or more, more preferably 400 or more, and more preferably 500 or more. The upper limit is preferably 1200 or less, more preferably 1000 or less, and still more preferably 900 or less. In a case where the molecular weight is 1200 or less, a vapor deposition temperature is not increased, and the compound is not easily decomposed. In a case where the molecular weight is 350 or more, a glass transition point of a vapor deposition film is not lowered, and the heat resistance of the photoelectric conversion element is improved.

The specific compound is particularly useful as a material of the photoelectric conversion film used for the imaging element, the optical sensor, or a photoelectric cell. The specific compound can also be used as a coloring material, a liquid crystal material, an organic semiconductor material, a charge transport material, a pharmaceutical material, and a fluorescent diagnostic material.

The maximum absorption wavelength of the specific compound is preferably, for example, within a range of 300 to 700 nm, and more preferably within a range of 400 to 600 nm.

The maximum absorption wavelength is a value measured in a solution state (solvent: chloroform) by an absorption spectrum of the specific compound being adjusted to a concentration having an absorbance of about 0.5 to 1. However, in a case where the specific compound is not dissolved in chloroform, a value measured by using the specific compound in which the specific compound is vapor-deposited and formed into a film state is defined as a maximum absorption wavelength of the specific compound.

The maximum absorption wavelength of the photoelectric conversion film is preferably, for example, within a range of 300 to 700 nm, and more preferably within a range of 400 to 700 nm.

The specific compound can also be used as a coloring agent, a p-type semiconductor material (material having excellent hole transport properties), and an n-type semiconductor material (material having excellent electron transport properties).

In a case where the specific compound is used as the p-type semiconductor material, the ionization potential of the specific compound is preferably 5.0 to 6.0 eV.

In addition, in a case where the specific compound is used as the n-type semiconductor material, the electron affinity of the specific compound is preferably 3.0 to 4.5 eV.

In the present specification, a value (value multiplied by -1) of a reciprocal number of the LUMO value obtained by the calculation of B3LYP/6-31G (d) using Gaussian′09 (software manufactured by Gaussian, Inc.) is used as a value of the electron affinity.

From the viewpoint of the responsiveness of the photoelectric conversion element, a content of the specific compound in the photoelectric conversion film (=(film thickness of specific compound in terms of single layer/film thickness of photoelectric conversion film) × 100) is preferably 15% to 85% by volume.

The photoelectric conversion element may contain one specific compound alone, or may contain two or more specific compounds.

### N-Type Semiconductor Material

The photoelectric conversion film preferably contains the n-type semiconductor material in addition to the specific compound.

The n-type semiconductor material is an acceptor-property organic semiconductor material (a compound), and refers to an organic compound having a property of easily accepting an electron.

More specifically, the n-type semiconductor material is preferably an organic compound having a higher electron affinity than that of the specific compound in a case where the n-type semiconductor material is used by being brought in contact with the above-described specific compound.

In addition, the n-type semiconductor material is preferably an organic compound having a higher electron affinity than the coloring agent in a case where the n-type semiconductor material is used by being brought in contact with the coloring agent described later.

The electron affinity of the n-type semiconductor material is preferably 3.0 to 5.0 eV.

Examples of the n-type semiconductor material include fullerenes selected from the group consisting of a fullerene and derivatives thereof, fused aromatic carbocyclic compounds (for example, a naphthalene derivative, an anthracene derivative, a phenanthrene derivative, a tetracene derivative, a pyrene derivative, a perylene derivative, and a fluoranthene derivative); a heterocyclic compound having a 5- to 7-membered ring having one or more selected from the group consisting of a nitrogen atom, an oxygen atom, or a sulfur atom (for example, pyridine, pyrazine, pyrimidine, pyridazine, triazine, quinoline, quinoxaline, quinazoline, phthalazine, cinnoline, isoquinoline, pteridine, acridine, phenazine, phenanthroline, tetrazole, pyrazole, imidazole, and thiazole); polyarylene compounds; fluorene compounds; cyclopentadiene compounds; silyl compounds; 1,4,5,8-naphthalenetetracarboxylic acid anhydride; 1,4,5,8-naphthalenetetracarboxylic acid anhydride imide derivative; oxadiazole derivative; anthraquinodimethane derivatives; diphenylquinone derivatives; bathocuproine, bathophenanthroline, and derivatives thereof; triazole compounds; a distyrylarylene derivative; a metal complex having a nitrogen-containing heterocyclic compound as a ligand; a silole compound; and compounds disclosed in paragraphs [0056] to [0057] of JP2006-100767A.

It is preferable that examples of the n-type semiconductor material include fullerenes selected from the group consisting of a fullerene and derivatives thereof.

Examples of the fullerenes include a fullerene C₆₀, a fullerene C₇₀, a fullerene C₇₆, a fullerene C₇₈, a fullerene C₈₀, a fullerene C₈₂, a fullerene C₈₄, a fullerene C₉₀, a fullerene C₉₆, a fullerene C₂₄₀, a fullerene C₅₄₀, and a mixed fullerene.

Examples of the fullerene derivatives include compounds in which a substituent is added to the above fullerenes. The substituent is preferably an alkyl group, an aryl group, or a heterocyclic group. As the fullerene derivative, the compounds described in JP2007-123707A are preferable.

In a case where the photoelectric conversion film contains the n-type semiconductor material, a content of the n-type semiconductor material in the photoelectric conversion film (=film thickness of n-type semiconductor material in terms of single layer/film thickness of photoelectric conversion film ×100) is preferably 15% to 75% by volume, more preferably 20% to 60% by volume, and still more preferably 25% to 50% by volume.

The n-type semiconductor material may be used alone, or two or more kinds thereof may be used in combination.

In addition, in a case where the n-type semiconductor material includes fullerenes, a content of the fullerenes to a total content of the n-type semiconductor material (=(film thickness of fullerenes in terms of single layer/total film thickness of n-type semiconductor materials in terms of single layer) ×100) is preferably 50% to 100% by volume, and more preferably 80% to 100% by volume.

The fullerenes may be used alone, or two or more kinds thereof may be used in combination.

The molecular weight of the n-type semiconductor material is preferably 200 to 1200, and more preferably 200 to 1000.

### P-Type Semiconductor Material

The photoelectric conversion film also preferably contains the p-type semiconductor material in addition to the specific compound.

The p-type semiconductor material is a donor organic semiconductor material (a compound), and refers to an organic compound having a property of easily donating an electron. Specifically, the p-type semiconductor material is preferably an organic compound having more excellent hole transport properties than a specific compound in the photoelectric conversion film, and is more preferably an organic compound having more excellent hole transport properties than any one of the specific compound or a coloring agent described later.

In the present specification, the hole transport properties (hole carrier mobility) of a compound can be evaluated by, for example, a time-of-flight method (such as a TOF method, for example) or by using a field effect transistor element.

The hole carrier mobility of the p-type semiconductor material is preferably 10⁻⁴ cm²/V·s or more, more preferably 10⁻³ cm²/V·s or more, and still more preferably 10⁻² cm²/V·s or more. The upper limit is preferably 10 cm²/V·s or less from the viewpoint of suppressing the flow of a small amount of current without light irradiation.

In addition, the p-type semiconductor material preferably has a smaller ionization potential than the specific compound in the photoelectric conversion film, and more preferably has a smaller ionization potential than any one of the specific compound or a coloring agent described later.

Examples of the p-type semiconductor material include triarylamine compounds (for example, N, N′-bis (3-methylphenyl)-(1,1′-biphenyl)-4,4′-diamine (TPD), 4,4′-bis [N-(naphthyl)-N-Phenyl-amino] biphenyl (α-NPD), compounds disclosed in paragraphs [0128] to [0148] of JP2011-228614A, compounds disclosed in paragraphs [0052] to [0063] of JP2011-176259A, compounds disclosed in paragraphs [0119] to [0158] of JP2011-225544A, compounds disclosed in [0044] to [0051] of JP2015-153910A, and compounds disclosed in paragraphs [0086] to [0090] of JP2012-094660A, pyrazoline compounds, styrylamine compounds, hydrazone compounds, polysilane compounds, thiophene compounds (for example, a thienothiophene derivative, a dibenzothiophene derivative, a benzodithiophene derivative, a dithienothiophene derivative, a [1] benzothieno [3,2-b] thiophene (BTBT) derivative, a thieno [3,2-f: 4,5-f] bis [1] benzothiophene (TBBT) derivative, compounds disclosed in paragraphs [0031] to [0036] of JP2018-14474A, compounds disclosed in paragraphs [0043] to [0045] of WO2016-194630A, compounds disclosed in paragraphs [0025] to [0037], and [0099] to [0109] of WO2017-159684A, compounds disclosed in paragraphs [0029] to [0034] of JP2017-076766A, compounds disclosed in paragraphs [0015] to [0025] of WO2018-207722A, compounds disclosed in paragraphs [0045] to [0053] of JP2019-54228A, compounds disclosed in paragraphs [0045] to [0055] of WO2019-058995A, compounds disclosed in paragraphs [0063] to [0089] of WO2019-081416A, compounds disclosed in paragraphs [0033] to [0036] of JP2019-80052A, compounds disclosed in paragraphs [0044] to [0054] of WO2019-054125A, compounds disclosed in paragraphs [0041] to [0046] of WO2019-093188A, and the like), a cyanine compound, an oxonol compound, a polyamine compound, an indole compound, a pyrrole compound, a pyrazole compound, a polyarylene compound, a fused aromatic carbocyclic compound (for example, a naphthalene derivative, an anthracene derivative, a phenanthrene derivative, a tetracene derivative, a pentacene derivative, a pyrene derivative, a perylene derivative, a fluoranthene derivative, and the like), a porphyrin compound, a phthalocyanine compound, a triazole compound, an oxadiazole compound, an imidazole compound, a polyarylalkane compound, a pyrazolone compound, an amino-substituted chalcone compound, an oxazole compound, a fluorenone compound, a silazane compound, and a metal complex having nitrogen-containing heterocyclic compounds as ligands.

In addition, the p-type semiconductor material is preferably a compound represented by any of Formula (p1) to Formula (p6), and more preferably a compound represented by Formula (p1).

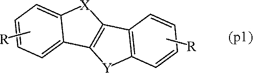

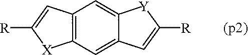

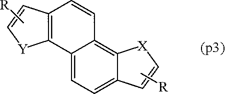

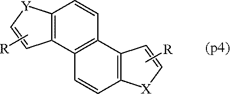

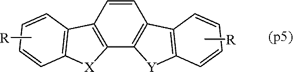

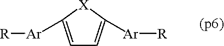

Two R’s present in Formulae (p1) to (p6) each independently represent a hydrogen atom or a substituent.

Examples of the substituent include an alkyl group, an alkoxy group, a halogen atom, an alkylthio group, a (hetero)arylthio group, an alkylamino group, a (hetero)arylamino group, and a (hetero)aryl group. The above-described substituents may further have a substituent. Specifically, an example of the (hetero)aryl group includes an arylaryl group, which may further have a substituent (that is, a biaryl group). In addition, at least one of the two aryl groups constituting the biaryl group may be a heteroaryl group.

The “(hetero)aryl group” is a concept including both an aryl group and a heteroaryl group. Specifically, in a case of a “(hetero)arylthio group”, any one of an arylthio group or a heteroarylthio group may be used.

In addition, groups represented by R in Formula (IX) described in WO2019/081416A are also preferably used as R.

X and Y each independently represent —CR²₂—, a sulfur atom (—S—), an oxygen atom (—O—), —NR²—, or —SiR²₂—.

R² represents a hydrogen atom, an alkyl group (preferably a methyl group or a trifluoromethyl group), an aryl group, or a heteroaryl group, which may have a substituent. Two or more R²′s may be the same or different from each other.

Ar represents an aromatic ring group. As Ar, a benzene ring group is preferable.

The above-described aromatic ring group may be a monocyclic ring or a polycyclic ring (for example, a bicyclic ring, a tricyclic ring, a tetracyclic ring, or the like).

In a case where the photoelectric conversion film contains the p-type semiconductor material, a content of the p-type semiconductor material in the photoelectric conversion film (=film thickness of p-type semiconductor material in terms of single layer/film thickness of photoelectric conversion film ×100) is preferably 15% to 75% by volume, more preferably 20% to 60% by volume, and still more preferably 25% to 50% by volume.

The p-type semiconductor material may be used alone, or two or more kinds thereof may be used in combination.

### Coloring Agent

The photoelectric conversion film may further include a coloring agent in addition to the specific compound.

The coloring agent is preferably an organic coloring agent.

Examples of the coloring agent include a cyanine coloring agent, a styryl coloring agent, a hemicyanine coloring agent, a merocyanine coloring agent (including zeromethine merocyanine (simple merocyanine)), a rhodacyanine coloring agent, an allopolar coloring agent, an oxonol coloring agent, a hemioxonol coloring agent, a squarylium coloring agent, a croconium coloring agent, an azamethine coloring agent, a coumarin coloring agent, an arylidene coloring agent, an anthraquinone coloring agent, a triphenylmethane coloring agent, an azo coloring agent, an azomethine coloring agent, a metallocene coloring agent, a fluorenone coloring agent, a flugide coloring agent, a perylene coloring agent, a phenazine coloring agent, a phenothiazine coloring agent, a quinone coloring agent, a diphenylmethane coloring agent, a polyene coloring agent, an acridine coloring agent, a quinoxaline coloring agent, an acridinone coloring agent, a diphenylamine coloring agent, a quinophthalone coloring agent, a phenoxazine coloring agent, a phthaloperylene coloring agent, a dioxane coloring agent, a porphyrin coloring agent, a chlorophyll coloring agent, a phthalocyanine coloring agent, a subphthalocyanine coloring agent, a metal complex coloring agent, compounds disclosed in paragraphs [0083] to [0089] of JP2014-082483A, compounds disclosed in paragraphs [0029] to [0033] of JP2009-167348A, compounds disclosed in paragraphs [0197] to [0227] of JP2012-077064A, compounds disclosed in paragraphs [0035] to [0038] of WO2018-105269A, compounds disclosed in paragraphs [0041] to [0043] of WO2018-186389A, compounds disclosed in paragraphs [0059] to [0062] of WO2018-186397A, compounds disclosed in paragraphs [0078] to [0083] of WO2019-009249A, compounds disclosed in paragraphs [0054] to [0056] of WO2019-049946A, compounds disclosed in paragraphs [0059] to [0063] of WO2019-054327A, compounds disclosed in paragraphs [0086] to [0087] of WO2019-098161A, and compounds disclosed in paragraphs [0085] to [0114] of WO2020-013246.

A content of the coloring agent in the photoelectric conversion film (=film thickness of coloring agent in terms of single layer/film thickness of photoelectric conversion film ×100) is preferably 1% to 85% by volume, more preferably 5% to 60% by volume, and still more preferably 10% to 40% by volume.

A content of the coloring agent with respect to the total content of the specific compound and the coloring agent in the photoelectric conversion film (=(film thickness of coloring agent in terms of single layer/(film thickness of specific compound in terms of single layer + film thickness of coloring agent in terms of single layer) ×100)) is preferably 1% to 75% by volume, more preferably 5% to 65% by volume, and still more preferably 10% to 60% by volume.

The coloring agent may be used alone, or two or more kinds thereof may be used in combination.

The maximum absorption wavelength of the coloring agent is preferably within a range of 400 to 700 nm, and more preferably within a range of 400 to 650 nm.

The maximum absorption wavelength is a value measured in a solution state (solvent: chloroform) by an absorption spectrum of the coloring agent being adjusted to a concentration having an absorbance of about 0.5 to 1. However, in a case where the coloring agent is not dissolved in chloroform, a value measured by using the coloring agent in which the coloring agent is vapor-deposited and formed into a film state is defined as a maximum absorption wavelength of the coloring agent.

The photoelectric conversion film is also substantially preferably composed of only the specific compound, the n-type semiconductor material, and the p-type semiconductor material. The “photoelectric conversion film is substantially composed of only the specific compound, the n-type semiconductor material, and the p-type semiconductor material” means the “total content of the specific compound, the n-type semiconductor material, and the p-type semiconductor material with respect to the total mass of the photoelectric conversion film is 95% to 100% by mass”.

The photoelectric conversion film is also substantially preferably composed of only the specific compound, the coloring agent, the n-type semiconductor material, and the p-type semiconductor material. The “photoelectric conversion film is substantially composed of only the specific compound, the coloring agent, the n-type semiconductor material, and the p-type semiconductor material” means the “total content of the specific compound, the coloring agent, the n-type semiconductor material, and the p-type semiconductor material with respect to the total mass of the photoelectric conversion film is 95% to 100% by mass”.

In addition, in a case where the photoelectric conversion film contains the n-type semiconductor material and/or the p-type semiconductor material, the photoelectric conversion film is preferably a mixture layer formed in a state where the specific compound, the n-type semiconductor material, and/or the p-type semiconductor material are mixed.

In addition, in a case where the photoelectric conversion film contains the coloring agent, the n-type semiconductor material, and/or the p-type semiconductor material, the photoelectric conversion film is preferably a mixture layer formed in a state where the specific compound, the coloring agent, the n-type semiconductor material, and/or the p-type semiconductor material are mixed.

The mixture layer is a layer in which two or more materials are mixed in a single layer.

The photoelectric conversion film containing the specific compound is a non-light emitting film, and has properties different from organic light emitting diodes (OLEDs). The non-light emitting film means a film having a light emission quantum efficiency of 1% or less, and the light emission quantum efficiency is preferably 0.5% or less, and more preferably 0.1% or less.

### Film Formation Method

Examples of a film formation method of the photoelectric conversion film include a method of forming a film by a dry film formation method.

Examples of the dry film formation method include a physical vapor deposition method such as a vapor deposition method (for example, a vacuum vapor deposition method or the like), a sputtering method, and an ion plating method, a molecular beam epitaxy (MBE) method, and a chemical vapor deposition (CVD) method such as plasma polymerization, and the vacuum vapor deposition method is preferable. In a case where the photoelectric conversion film is formed by the vacuum vapor deposition method, manufacturing conditions such as a degree of vacuum and a vapor deposition temperature can be set according to the normal method.

The thickness of the photoelectric conversion film is preferably 10 to 1000 nm, more preferably 50 to 800 nm, still more preferably 50 to 500 nm, and particularly preferably 50 to 400 nm.

### Electrode (Conductive Film)

Electrodes (the upper electrode (the transparent conductive film) 15 and the lower electrode (the conductive film) 11) are formed of conductive materials. Examples of the conductive material include metals, alloys, metal oxides, electrically conductive compounds, and mixtures thereof.

Since light is incident through the upper electrode 15, the upper electrode 15 is preferably transparent to light to be detected. Examples of the materials constituting the upper electrode 15 include conductive metal oxides such as tin oxide (antimony tin oxide (ATO) or fluorine doped tin oxide (FTO)) doped with antimony, fluorine or the like, tin oxide, zinc oxide, indium oxide, indium tin oxide (ITO), and indium zinc oxide (IZO); metal thin films such as gold, silver, chromium, and nickel; mixtures or laminates of these metals and the conductive metal oxides; organic conductive materials such as polyaniline, polythiophene, and polypyrrole; and carbon materials such as graphene and carbon nanotubes, and conductive metal oxides are preferable in terms of high conductivity and transparency.

In general, in a case where the conductive film is made to be thinner than a certain range, a resistance value is rapidly increased. By contrast, in the solid-state imaging element into which the photoelectric conversion element according to the present embodiment is incorporated, the sheet resistance is, for example, 100 to 10000 Ω/□, and a degree of freedom of a range of the film thickness that can be thinned is large. In addition, as the thickness of the upper electrode (the transparent conductive film) 15 is thinner, the amount of light that the upper electrode absorbs is smaller, and the light transmittance usually increases. The increase in the light transmittance causes an increase in light absorbance in the photoelectric conversion film and an increase in the photoelectric conversion ability, which is preferable. Considering the suppression of leakage current, an increase in the resistance value of the thin film, and an increase in transmittance accompanied by the thinning, the film thickness of the upper electrode 15 is preferably 5 to 100 nm, and more preferably 5 to 20 nm.

There is a case where the lower electrode 11 has transparency or an opposite case where the lower electrode 11 does not have transparency and reflects light, depending on the application. Examples of a material constituting the lower electrode 11 include conductive metal oxides such as tin oxide (ATO or FTO) doped with antimony, fluorine, or the like, tin oxide, zinc oxide, indium oxide, indium tin oxide (ITO), and indium zinc oxide (IZO); metals such as gold, silver, chromium, nickel, titanium, tungsten, and aluminum, and conductive compounds (for example, titanium nitride (TiN)) such as oxides or nitrides of these metals; mixtures or laminates of these metals and conductive metal oxides; organic conductive materials such as polyaniline, polythiophene, and polypyrrole; carbon materials such as graphene and carbon nanotubes.

The method of forming electrodes can be appropriately selected in accordance with the electrode material. Specific examples thereof include a wet method such as a printing method and a coating method; a physical method such as a vacuum vapor deposition method, a sputtering method, and an ion plating method; and a chemical method such as a CVD method and a plasma CVD method.

In a case where the material of the electrode is ITO, examples thereof include an electron beam method, a sputtering method, a resistance heating vapor deposition method, a chemical reaction method (for example, a sol-gel method or the like), and a coating method with a dispersion of indium tin oxide.

### Charge Blocking Film: Electron Blocking Film and Hole Blocking Film

It is also preferable that the photoelectric conversion element according to the embodiment of the present invention has one or more interlayers between the conductive film and the transparent conductive film, in addition to the photoelectric conversion film.

An example of the interlayer includes a charge blocking film. In a case where the photoelectric conversion element has the above-described film, the characteristics (for example, photoelectric conversion efficiency, responsiveness, and the like) of the obtained photoelectric conversion element are more excellent. Examples of the charge blocking film include an electron blocking film and a hole blocking film. Hereinafter, each of the films will be described in detail.

### Electron Blocking Film

The electron blocking film is a donor organic semiconductor material (compound).

In addition, as the donor organic semiconductor material, the above-described p-type organic semiconductor can also be used. The p-type organic semiconductor may be used alone, or two or more kinds thereof may be used in combination.

Examples of the p-type organic semiconductor used in the electron blocking film include compounds having a smaller ionization potential than that of the n-type semiconductor material, and in a case where this condition is satisfied, the above-described coloring agents can be used.

A polymer material can also be used as the electron blocking film.

Examples of the polymer material include a polymer such as phenylenevinylene, fluorene, carbazole, indole, pyrene, pyrrole, picoline, thiophene, acetylene, and diacetylene, and a derivative thereof.

The electron blocking film may be formed of a plurality of films.

The electron blocking film may be formed of an inorganic material. In general, since an inorganic material has a dielectric constant larger than that of an organic material, in a case where the inorganic material is used in the electron blocking film, a large voltage is applied to the photoelectric conversion film. Therefore, the photoelectric conversion efficiency increases. Examples of the inorganic material that can be used for the electron blocking film include calcium oxide, chromium oxide, copper chromium oxide, manganese oxide, cobalt oxide, nickel oxide, copper oxide, copper gallium oxide, copper strontium oxide, niobium oxide, molybdenum oxide, copper indium oxide, silver indium oxide, and iridium oxide.

### Positive Hole Blocking Film

The hole blocking film is an acceptor-property organic semiconductor material (compound).

As the acceptor-property organic semiconductor material, the above-described n-type semiconductor material can also be used.

Examples of a method of producing a charge blocking film include a dry film formation method and a wet film formation method.

Examples of the dry film formation method include a vapor deposition method and a sputtering method. The vapor deposition method may be any of a physical vapor deposition (PVD) method and a chemical vapor deposition (CVD) method, and the physical vapor deposition method such as a vacuum vapor deposition method is preferable. Examples of the wet film formation method include an ink jet method, a spray method, a nozzle printing method, a spin coating method, a dip coating method, a casting method, a die coating method, a roll coating method, a bar coating method, and a gravure coating method, and an ink jet method is preferable from the viewpoint of high accuracy patterning.

Each thickness of the charge blocking films (the electron blocking film and the positive hole blocking film) is preferably 3 to 200 nm, more preferably 5 to 100 nm, and still more preferably 5 to 30 nm.

### Substrate

The photoelectric conversion element may further include a substrate.

Examples of the above-described substrate include a semiconductor substrate, a glass substrate, and a plastic substrate.

In the photoelectric conversion element, a position of the substrate is not particularly limited, and in general, the conductive film, the photoelectric conversion film, and the transparent conductive film are laminated on the substrate in this order.

### Sealing Layer

The photoelectric conversion element may further include a sealing layer.

The performance of a photoelectric conversion material may deteriorate noticeably due to the presence of deterioration factors such as water molecules. The deterioration can be prevented by coating and sealing the entirety of the photoelectric conversion film with the sealing layer such as diamond-like carbon (DLC) or ceramics such as metal oxide, or metal nitride, and metal nitride oxide which are dense and into which water molecules do not permeate.

The material of the sealing layer may be selected and the sealing layer may be manufactured according to the description in paragraphs [0210] to [0215] of JP2011-082508A.

### Imaging Element and Optical Sensor

An example of the application of the photoelectric conversion element includes an imaging element.

The imaging element is an element that converts optical information of an image into an electric signal. In general, a plurality of the photoelectric conversion elements are arranged in a matrix on the same plane, and an optical signal is converted into an electric signal in each photoelectric conversion element (pixel) to sequentially output the electric signal to the outside of the imaging element for each pixel. Therefore, each pixel is formed of one or more photoelectric conversion elements and one or more transistors.

The imaging element is mounted on an imaging element such as a digital camera and a digital video camera, an electronic endoscope, and imaging modules such as a cellular phone.

The photoelectric conversion element according to the embodiment of the present invention is also preferably used for an optical sensor including the photoelectric conversion element of the present invention.

The photoelectric conversion element may be used alone as the optical sensor, and the photoelectric conversion element may be used as a line sensor in which the photoelectric conversion elements are linearly arranged or as a two-dimensional sensor in which the photoelectric conversion elements are arranged in a plane shape.

### Compound

The present invention also relates to a compound.

The compound according to the embodiment of the present invention has the same definition as the specific compound described above, and the suitable embodiments thereof are also the same.

### EXAMPLES

The present invention will be described in more detail below based on Examples. Materials, used amounts, ratios, treatment contents, treatment procedures, and the like described in the following Examples can be appropriately changed within the range that does not depart from the gist of the present invention. Therefore, the range of the present invention should not be limitatively interpreted by the following Examples.

### Compound (Compound for Evaluation)

### Synthesis Method of Compounds (2-24) and (2-25)

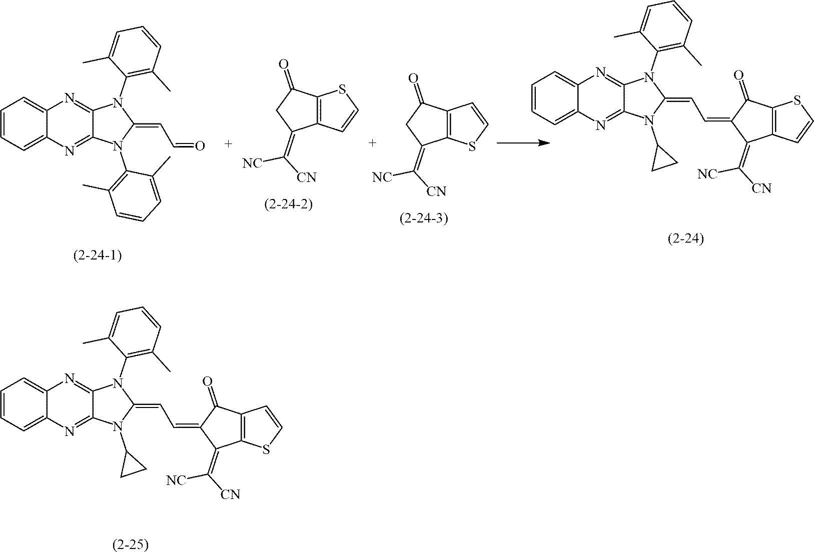

### Synthesis of Compound (2-24) and Compound (2-25)

5 mmol of a compound (2-24-1), 6.0 mmol of a mixture of a compound (2-24-2) and a compound (2-24-3), and 30 mL of acetic anhydride were added to a glass reaction container, and a reaction was carried out at 110° C. for 3 hours under a nitrogen atmosphere. Methanol was added to a reaction solution, and a solid was collected by filtration.

The obtained solid was washed with tetrahydrofuran (THF) and toluene, and separated into a compound (2-24) and a compound (2-25) by silica gel column chromatography. Each of the compounds was sublimated and purified to obtain 2.5 mmol of the compound (2-24) and 1.0 mmol of the compound (2-25).

### Compound (2-24):

¹H-NMR (CDCl₃, 400 MHz) δ = 1.35(2H,s), 1.65(2H,s), 2.09(6H,s), 3.76(1H,s), 7.07(1H,d), 7.37(2H,d), 7.50(1H,t), 7.59-7.68(2H,m), 7.71(1H,d), 7.82(1H,s), 7.88(1H,dd), 8.08(1H,d), 9.11(1H,d).

### Compound (2-25):

¹H-NMR (CDCl₃,400 MHz) δ = 1.35(2H,s), 1.66(2H,s), 2.09(6H,s), 3.75(1H,s), 7.04(1H,d), 7.20(1H,d), 7.38(2H,d), 7.50(1H,t), 7.59-7.68(3H,m), 7.88(1H,d), 8.08(1H,d), 9.10(1H,d).

Other specific compounds were also synthesized with reference to the above-described synthesis method.

The specific compound and comparative compound used in a test are shown below.

Hereinbelow, compounds (1-1) to (1-10) and compounds (2-1) to (2-32) are specific compounds.

Hereinafter, the specific compound and the Comparative compound are collectively referred to as a compound for evaluation.

Evaluation compounds were used for producing photoelectric conversion elements described later.

### Specific Compound

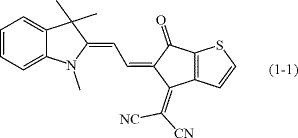

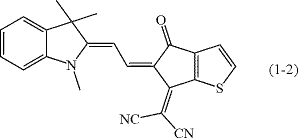

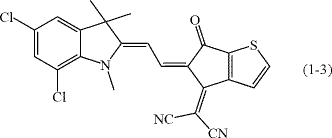

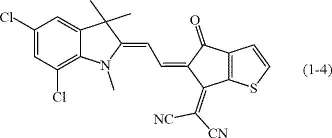

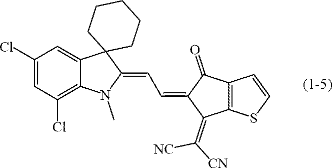

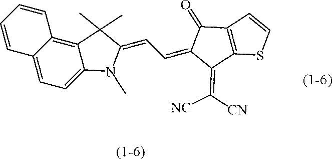

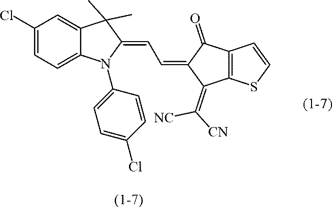

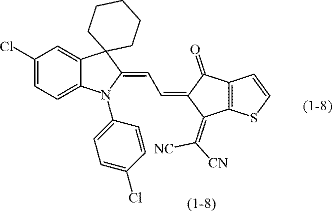

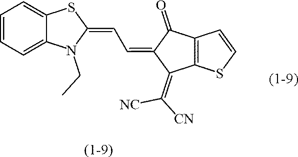

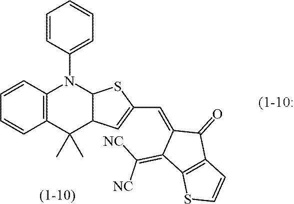

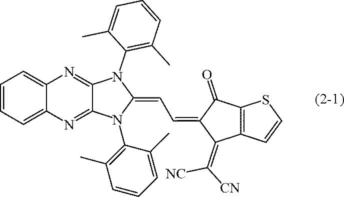

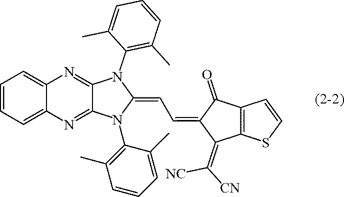

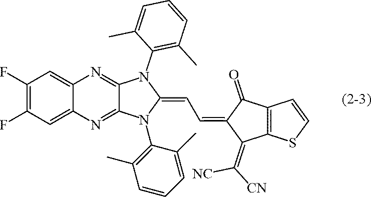

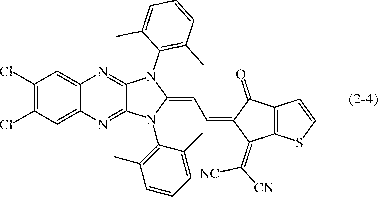

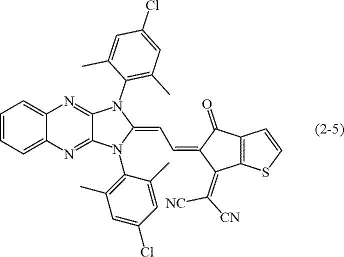

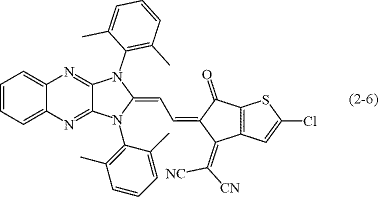

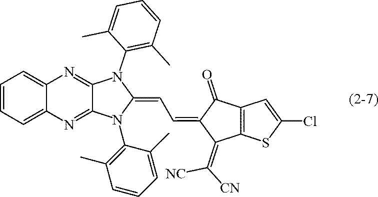

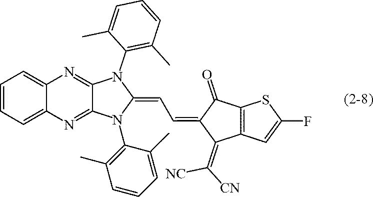

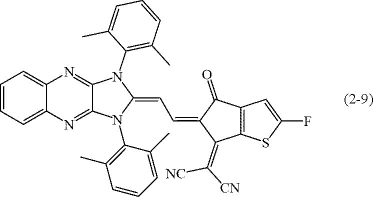

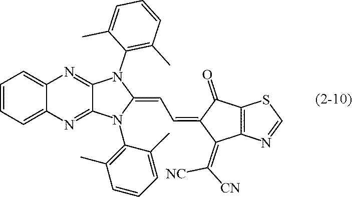

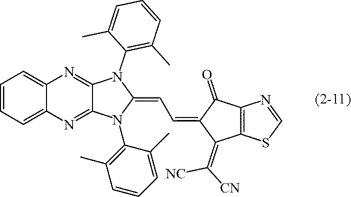

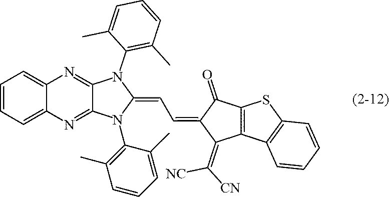

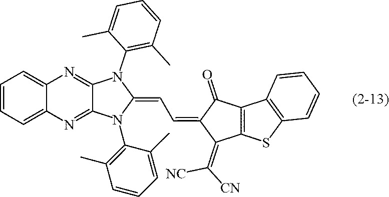

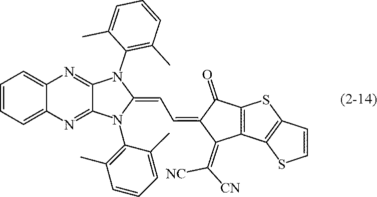

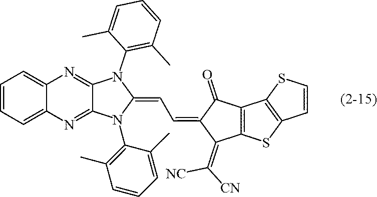

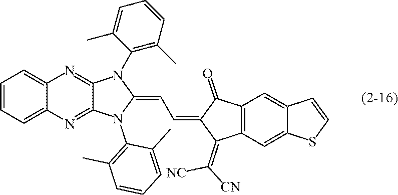

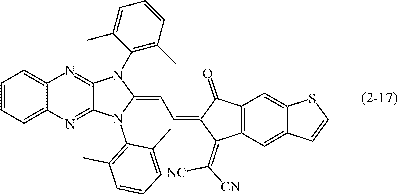

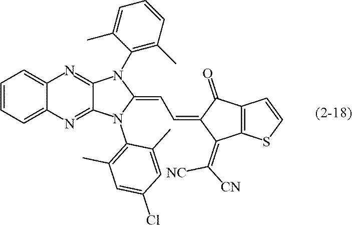

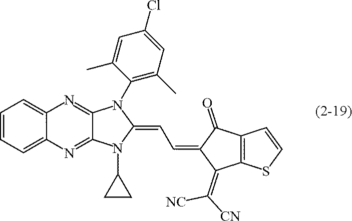

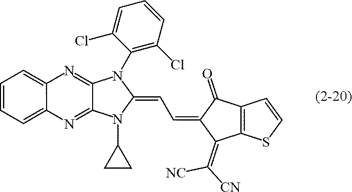

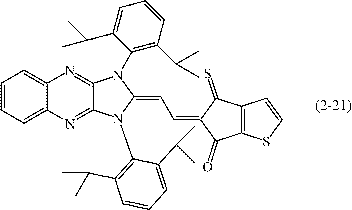 [SMILES: CC(C)c1cccc(C(C)C)c1N1C(=C/C=C2/C(=O)c3sccc3C2=S)N(c2c(C(C)C)cccc2C(C)C)c2nc3ccccc3nc21]

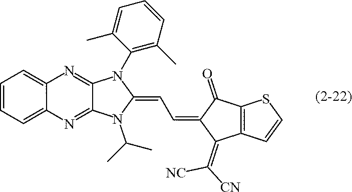

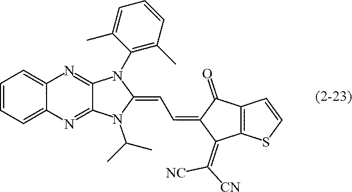

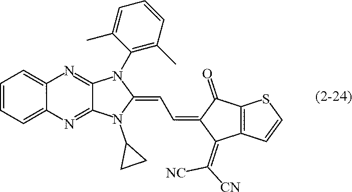

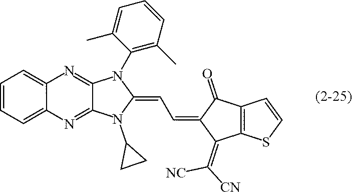

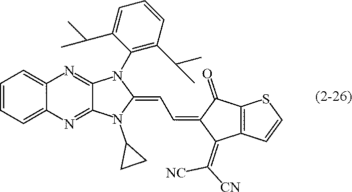

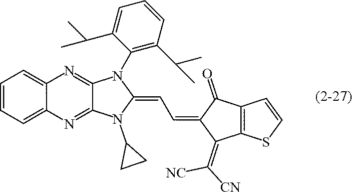

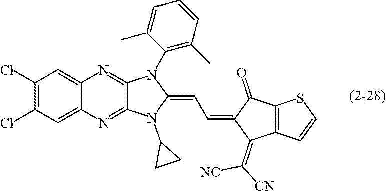

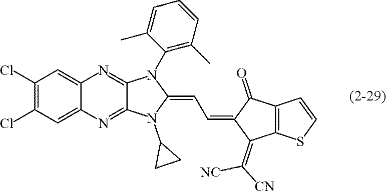

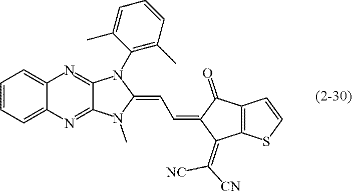

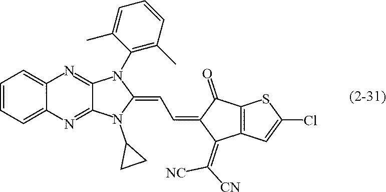

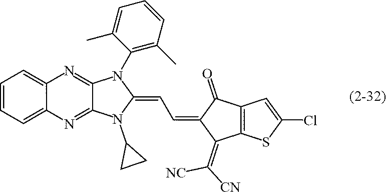

### Comparative Compound

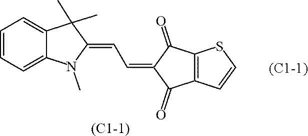

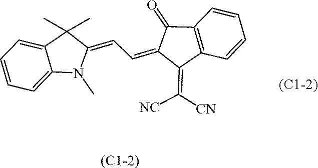

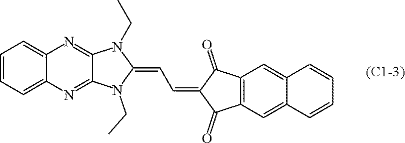 [SMILES: CCN1C(=CC=C2C(=O)c3cc4ccccc4cc3C2=O)N(CC)c2nc3ccccc3nc21]

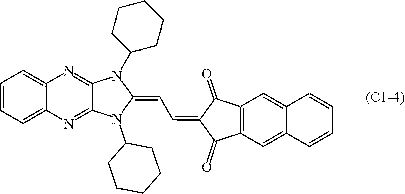 [SMILES: O=C1C(=CC=C2N(C3CCCCC3)c3nc4ccccc4nc3N2C2CCCCC2)C(=O)c2cc3ccccc3cc21]

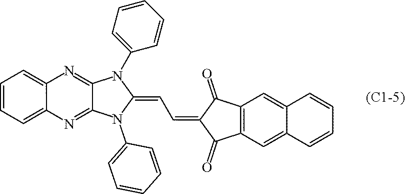 [SMILES: O=C1C(=CC=C2N(c3ccccc3)c3nc4ccccc4nc3N2c2ccccc2)C(=O)c2cc3ccccc3cc21]

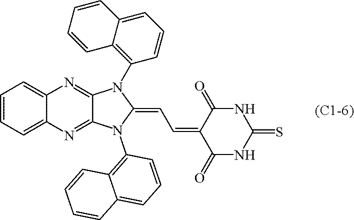 [SMILES: O=C1NC(=S)NC(=O)C1=CC=C1N(c2cccc3ccccc23)c2nc3ccccc3nc2N1c1cccc2ccccc12]

### P-Type Semiconductor

The p-type semiconductor described below was used for producing the photoelectric conversion elements described later, as the p-type semiconductor used for evaluations.

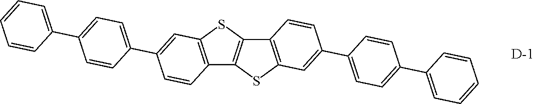 [SMILES: c1ccc(-c2ccc(-c3ccc4c(c3)sc3c5ccc(-c6ccc(-c7ccccc7)cc6)cc5sc43)cc2)cc1]

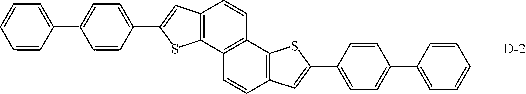 [SMILES: c1ccc(-c2ccc(-c3cc4ccc5c(ccc6cc(-c7ccc(-c8ccccc8)cc7)sc65)c4s3)cc2)cc1]

### N-Type Semiconductor

Fullerene C₆₀ was used for the production of photoelectric conversion elements described later, as the n-type semiconductor used for evaluations.

### Coloring Agent

In addition, in the production of a photoelectric conversion element (B) described later, the following coloring agents were used as the coloring agents used for the evaluation.

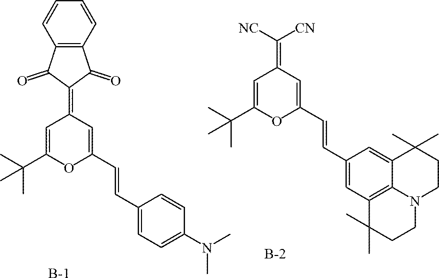

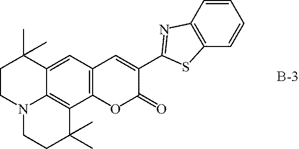 [SMILES: CC1(C)CCN2CCC(C)(C)c3c2c1cc1cc(-c2nc4ccccc4s2)c(=O)oc31]

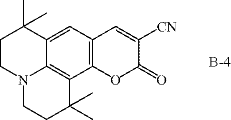 [SMILES: CC1(C)CCN2CCC(C)(C)c3c2c1cc1cc(C#N)c(=O)oc31]

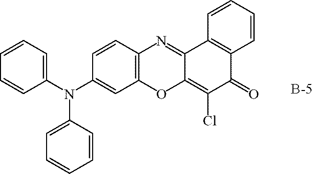 [SMILES: O=c1c(Cl)c2oc3cc(N(c4ccccc4)c4ccccc4)ccc3nc-2c2ccccc12]

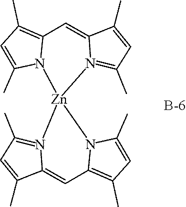

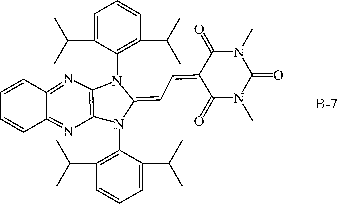 [SMILES: CC(C)c1cccc(C(C)C)c1N1C(=CC=C2C(=O)N(C)C(=O)N(C)C2=O)N(c2c(C(C)C)cccc2C(C)C)c2nc3ccccc3nc21]

### Evaluation

### Production of Photoelectric Conversion Element (A)

A photoelectric conversion element having the form illustrated in FIG. 2 was produced using evaluation compounds (specific compounds or comparative compounds). Here, the photoelectric conversion element includes a lower electrode 11, an electron blocking film 16A, a photoelectric conversion film 12, a positive hole blocking film 16B, and an upper electrode 15.

Specifically, an amorphous ITO was formed into a film on a glass substrate by a sputtering method to form the lower electrode 11 (thickness: 30 nm). Furthermore, a compound (B-1) described below was formed into a film on the lower electrode 11 by a vacuum thermal vapor deposition method to form the electron blocking film 16A (thickness: 30 nm). Furthermore, each material (evaluation compounds, p-type semiconductor, and n-type semiconductor (fullerene (C₆₀)) illustrated in Table 1 is vapor-deposited on the electron blocking film 16A to have a speed ratio illustrated in Table 1 to form a photoelectric conversion film 12 having a bulk hetero structure. Furthermore, a compound (B-2) described below was vapor-deposited on the photoelectric conversion film 12 to form the positive hole blocking film 16B (thickness: 10 nm). Amorphous ITO was formed into a film on the positive hole blocking film 16B by a sputtering method to form the upper electrode 15 (the transparent conductive film) (thickness: 10 nm). After the SiO film was formed as the sealing layer on the upper electrode 15 by a vacuum vapor deposition method, an aluminum oxide (Al₂O₃) layer was formed thereon by an atomic layer chemical vapor deposition (ALCVD) method to produce a photoelectric conversion element.

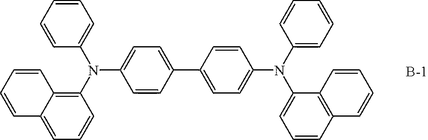 [SMILES: c1ccc(N(c2ccc(-c3ccc(N(c4ccccc4)c4cccc5ccccc45)cc3)cc2)c2cccc3ccccc23)cc1]

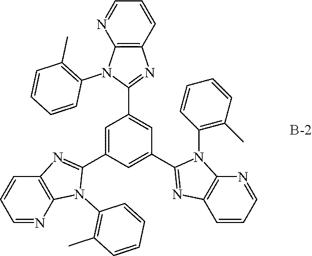 [SMILES: Cc1ccccc1-n1c(-c2cc(-c3nc4cccnc4n3-c3ccccc3C)cc(-c3nc4cccnc4n3-c3ccccc3C)c2)nc2cccnc21]

### Evaluation of Photoelectric Conversion Element (A)

### Evaluation of Photoelectric Conversion Efficiency

The drive of each of the photoelectric conversion elements obtained in Examples and Comparative Examples was confirmed.

A voltage was applied to each photoelectric conversion element so that an electric field strength thereof was 2.0 × 10⁵ V/cm, and light is emitted from an upper electrode (transparent conductive film) side to measure an integrated value (“integrated value of photoelectric conversion efficiency before annealing”) of the photoelectric conversion efficiency (external quantum efficiency) in a wavelength range of 450 to 650 nm. Thereafter, the element was heated in a glove box at 150° C. for 30 minutes, and the integrated value (“integrated value of photoelectric conversion efficiency after annealing”) of the photoelectric conversion efficiency (external quantum efficiency) in the wavelength range of 450 to 650 nm was then measured again.

The following evaluations (evaluation 1 and evaluation 2) were carried out based on the obtained measured values.

### Evaluation 1: Photoelectric Conversion Efficiency After Annealing

The relative photoelectric conversion efficiency was calculated by Expression (S1) described below, and the evaluation was carried out based on the following evaluation standard. The results are illustrated in Table 1.

$$\begin{matrix}\text{Relative photoelectric conversion efficiency =} \left(\text{integrated value of photoelectric}\right) \\ \text{conversion efficiency of each photoelectric conversion element of Examples or Comparative} \\ \left(\text{Examples in wavelength range of 450 to 650 nm after annealing}\right) \text{/} \left(\text{integrated value of}\right) \\ \text{photoelectric conversion efficiency of photoelectric conversion element of Example 1-1 in} \\ \left(\text{wavelength range of 450 to 650 after annealing}\right)\end{matrix}$$

### Evaluation Standard

“AA”: The variation value of the photoelectric conversion efficiency is 1.5 or more.

“A”: The relative photoelectric conversion efficiency is 1.3 or more and less than 1.5.

“B”: The relative photoelectric conversion efficiency is 1.1 or more and less than 1.3.

“C”: The relative photoelectric conversion efficiency is 0.9 or more and less than 1.1.

“D”: The relative photoelectric conversion efficiency is 0.7 or more and less than 0.9.

“E”: The relative photoelectric conversion efficiency is less than 0.7.

### Evaluation 2: Variation of Photoelectric Conversion Efficiency by Annealing

The variation value of the photoelectric conversion efficiency was calculated by Expression (S2) described below, and the evaluation was carried out based on the following evaluation standard. The results are illustrated in Table 1.

$$\begin{matrix}\text{Variation value of photoelectric conversion efficiency =} \left(\text{integrated value of}\right) \\ \text{photoelectric conversion efficiency of each photoelecric conversion element of Examples or} \\ \left(\text{Comparative Examples in wavelength range of 450 to 650 nm after annealing}\right) \text{/} \left(\text{integrated value}\right) \\ \text{of photoelectric conversion efficiency of each photoelectric conversion element of Examples or} \\ \left(\text{Comparative Examples in wavelength range of 450 to 650 nm before annealing}\right)\end{matrix} \qquad \text{Expression (S2):}$$

### Evaluation Standard

“A”: The variation value of the photoelectric conversion efficiency is 1.1 or more.

“B”: The variation value of the photoelectric conversion efficiency is 1.0 or more and less than 1.1.

“C”: The variation value of the photoelectric conversion efficiency is 0.9 or more and less than 1.0.

“D”: The variation value of the photoelectric conversion efficiency is 0.6 or more and less than 0.9.

“E”: The variation value of the photoelectric conversion efficiency is less than 0.6.

### Evaluation of Responsiveness

The evaluation of responsiveness was carried out by using each of the photoelectric conversion elements of Examples and Comparative Examples.

### Evaluation 3: Evaluation of Response Speed

A voltage was applied to each photoelectric conversion element to have a strength of 2.0 × 10⁵ V/cm. Thereafter, light emitting diodes (LEDs) were instantaneously turned on to emit light from an upper electrode (transparent conductive film) side, and a photocurrent at that time was measured with an oscilloscope. At this time, assuming that the current intensity (signal intensity) before light emission was 0%, and the maximum signal intensity measured by light emission was 100%, a time (rise time) until the signal intensity reached from 0% to 97% after light was emitted was determined for each photoelectric conversion element. Next, the relative response speed was calculated by Expression (S3) described below, and the evaluation was carried out based on the following evaluation standard. The results are illustrated in Table 1.

$$\begin{matrix}\text{Relative response speed =} \left(\text{rise time of each photoelectric conversion element of}\right) \\ \left(\text{Examples and Comparative Examples}\right) \text{/} \left(\text{rise time of photoelectric conversion element of}\right) \\ \left(\text{Example 1-1}\right)\end{matrix} \qquad \text{Expression (S3):}$$

### Evaluation Standard

“A”: The relative response speed is less than 0.5.

“B”: The relative response speed is 0.5 or more and less than 1.0.

“C”: The relative response speed is 1.0 or more and less than 1.5.

“D”: The relative response speed is 1.5 or more and less than 2.0.

“E”: The relative response speed is 2.0 or more.

### Evaluation 4: Evaluation of Electric Field Strength Dependence of Response Speed

A voltage was applied to each photoelectric conversion element to have a strength of 7.5 × 10⁴ V/cm. Thereafter, LEDs were instantaneously turned on to emit light from an upper electrode (transparent conductive film) side, and a photocurrent at that time was measured with an oscilloscope. At this time, assuming that the current intensity (signal intensity) before light emission was 0%, and the maximum signal intensity measured by light emission was 100%, a time (rise time) until the signal intensity reached from 0% to 97% after light emission was determined for each photoelectric conversion element. Next, the electric field strength dependence of the response speed was calculated by Expression (S4) described below, and the evaluation was carried out based on the following evaluation standard. The value of the denominator of Expression (S4) is a value obtained according to the procedure of the evaluation 3 described above. The results are illustrated in Table 1.

$$\begin{matrix}\text{Electric field strength dependence of relative speed = (rise time of each} \\ \text{photoelectric conversion element of Examples or Comparative Examples in a case where} \\ \text{voltage is applied to have intensity of 7} \text{.5} \times {\text{10}}^{\text{4}} \text{V/cm)/(rise time of each photoelectric} \\ \text{conversion element of Examples or Comparative Examples in a case where voltage is applied} \\ \text{to have intensity of 2} \text{.0} \times {\text{10}}^{\text{5}} \text{V/cm)}\end{matrix} \qquad \text{Expression (S4):}$$

### Evaluation Standard

“A”: The electric field strength dependence of the relative response is less than 2.0.

“B”: The electric field strength dependence of the relative response is 2.0 or more and less than 3.0.

“C”: The electric field strength dependence of the relative response is 3.0 or more and less than 4.0.

“D”: The electric field strength dependence of the relative response was 4.0 or more and less than 5.0.

“E”: The electric field strength dependence of the relative response is 5.0 or more.

Table 1 is illustrated below.

In Tables 1 and 2, “Structure of D¹” in the remarks column indicates the kind of the structure of D¹ in the specific compound. A case where D¹ in the specific compound represents a group represented by Formula (1D) described above is denoted by “1D”, a case where D¹ in the specific compound represents a group represented by Formula (2D) described above is denoted by “2D”, and a case where D¹ in the specific compound represents a group that does not correspond to any one of Formula (1D) or Formula (2D) is denoted by “N”.

In addition, in Tables 1 and 2, “Structure of A¹” in the remarks column indicates the kind of the structure of A¹ in the specific compound. A case where A¹ in the specific compound represents a group represented by Formula (1A) described above is denoted by “1A”, and a case where A¹ in the specific compound represents a group represented by Formula (2A) described above is denoted by “2A”.

In addition, in Tables 1 and 2, “Combination of Y¹¹/Y¹² and Y²¹/Y²²” in the remarks column indicates “Combination of Y¹¹ and Y¹²” in a case where A¹ in the specific compound represents a group represented by Formula (1A) described above, and indicates “Combination of Y²¹ and Y²²” in a case where A¹ in the specific compound represents a group represented by Formula (2A) described above. “O atom” means an oxygen atom, “S atom” means a sulfur atom, and “CN*” means ═C(CN)₂.

In addition, in Tables 1 and 2, “Asymmetry of R^{d11}/R^{d12}” in the remarks column indicates whether R^{d11} and R^{d12} are the same as each other or not, in a case where D¹ in the specific compound represents a group represented by the above-described Formula (1D). A case where R^{d11} and R^{d12} are not the same as each other is represented by “P”, and a case where R^{d11} and R^{d12} are the same as each other is represented by “N”. In addition, “-” means a case where D¹ in the specific compound does not correspond to the group represented by the above-described Formula (1D).

In addition, in Tables 1 and 2, “Structure of X^{d21}/R^{d22}”, in the remarks column indicates whether at least one or more requirements X1 to X3 described above are satisfied or not, in a case where D¹ in the specific compound represents a group represented by Formula (6D) described above. A case where at least one or more requirements X1 to X3 described above are satisfied is represented by “P”, and a case where at least one or more requirements X1 to X3 described above are not satisfied is represented by “N”. “-” means a case where D¹ in the specific compound does not correspond to the group represented by Formula (6D) described above.

In addition, in Tables 1 and 2, “Presence or absence of F atom in D¹ or A¹” in the remarks column indicates whether at least one of D¹ or A¹ in the specific compound contains a fluorine atom, and a case where the fluorine atom is not contained is represented by “P”, and a case where the fluorine atom is contained is represented by “N”.

In addition, in Tables 1 and 2, “Kind of A¹” in the remarks column indicates whether or not Y¹¹ represents an oxygen atom, and Y¹² represents ═C(CN)₂ in a case where A¹ in the specific compound is a group represented by Formula (laA), and X¹¹ represents —CR^{x}═ and X¹² represents a sulfur atom (but, a case where R^{x}’s in Formula (1Aa) are bonded to each other to form a ring is excluded). In a case where Y¹¹ represents an oxygen atom and Y¹² represents =C(CN)₂ is denoted by “P”, and in a case where Y¹¹ does not represent an oxygen atom and Y¹² does not represent ═C(CN)₂ is denoted by “N”.

TABLE 1

| Table 1 (Table 1-1) | Each component used to form photoelectric conversion film | | | | Remarks | | | | | | | | Evaluation result | | | |
|---|---|---|---|---|---|---|---|---|---|---|---|---|---|---|---|---|
| | Kind | | | Component ratio⁴ | Structure of D¹ | Structure of A¹ | Combination of Y¹¹/Y¹² and Y²¹/Y²² | | Asymmetry of R^{d11}/R^{d12} | Structure of X^{d21}/R^{d22} | Presence or absence of F atom in D¹ or A¹ | Kind of A¹ | Evaluation 1 | Evaluation 2 | Evaluation 3 | Evaluation 4 |
| | Evaluation compound | p-type semiconductor material | a-type semiconductor material | | | | | | | | | | Photoelectric conversion efficiency after annealing | Variation of photoelectric conversion efficiency by annealing | Response speed | Electric field strength dependence of response speed |
| Example 1-1 | 1-1 | D-1 | C₆₀ | 1:1:1 | 2D | 1A | O atom | CN* | - | N | P | N | C | C | C | C |
| Example 1-2 | 1-1 | D-2 | C₆₀ | 1:1:1 | 2D | 1A | O atom | CN* | - | N | P | N | C | C | C | C |
| Example 1-3 | 1-2 | D-1 | C₆₀ | 1:1:1 | 2D | 1A | O atom | CN* | - | N | P | P | B | C | B | C |
| Example 1-4 | 1-3 | D-1 | C₆₀ | 1:1:1 | 2D | 1A | O atom | CN* | - | N | P | N | C | C | C | C |
| Example 1-5 | 1-4 | D-1 | C₆₀ | 1:1:1 | 2D | 1A | O atom | CN* | - | N | P | P | B | C | B | C |
| Example 1-6 | 1-5 | D-1 | C₆₀ | 1:1:1 | 2D | 1A | O atom | CN* | - | P | P | P | A | B | A | B |
| Example 1-7 | 1-6 | D-1 | C₆₀ | 1:1:1 | 2D | 1A | O atom | CN* | - | P | P | P | A | B | A | B |
| Example 1-8 | 1-7 | D-1 | C₆₀ | 1:1:1 | 2D | 1A | O atom | CN* | - | P | P | P | A | B | A | B |
| Example 1-9 | 1-8 | D-1 | C₆₀ | 1:1:1 | 2D | 1A | O atom | CN* | - | P | P | P | A | B | A | B |
| Example 1-10 | 1-9 | D-1 | C₆₀ | 1:1:1 | 2D | 1A | O atom | CN* | - | N | P | P | B | C | B | C |
| Example 1-11 | 1-10 | D-1 | C₆₀ | 1:1:1 | N | 1A | O atom | CN* | - | - | P | P | B | C | B | C |
| Example 1-12 | 2-1 | D-1 | C₆₀ | 1:1:1 | 1D | 1A | O atom | CN* | N | - | P | N | B | A | A | A |
| Example 1- 13 | 2-2 | D-1 | C₆₀ | 1:1:1 | 1D | 1A | O atoms | CN* | N | - | P | P | A | A | A | A |
| Example 1-14 | 2-3 | D-1 | C₆₀ | 1:1:1 | 1D | 1A | O atom | CN* | N | - | N | P | B | A | B | A |
| Example 1-15 | 2-4 | D-1 | C₆₀ | 1:1:1 | 1D | 1A | O atom | CN* | N | - | P | P | A | A | A | A |
| Example 1-16 | 2-5 | D-1 | C₆₀ | 1:1:1 | 1D | 1A | O atom | CN* | N | - | P | P | A | A | A | A |
| Example 1-17 | 2-6 | D-1 | C₆₀ | 1:1:1 | 1D | 1A | O atom | CN* | N | - | P | N | B | A | A | A |
| Example 1-18 | 2-7 | D-1 | C₆₀ | 1:1:1 | 1D | 1A | O atom | CN* | N | - | P | P | A | A | A | A |
| Example 1-19 | 2-8 | D-1 | C₆₀ | 1:1:1 | 1D | 1A | O atom O atom | CN* | N | - | N | N | B | A | B | A |
| Example 1-20 | 2-9 | D-1 | C₆₀ | 1:1:1 | 1D | 1A | O atom | CN* | N | - | N | P | B | A | B | A |
| Example 1-21 | 2-10 | D-1 | C₆₀ | 1:1:1 | 1D | 1A | O atom | CN* | N | - | P | - | B | A | A | A |
| Example 1-22 | 2-11 | D-1 | C₆₀ | 1:1:1 | 1D | 1A | O atom | CN* | N | - | P | - | B | A | A | A |
| Example 1-23 | 2-12 | D-1 | C₆₀ | 1:1:1 | 1D | 1A | O atom | CN* | N | - | P | - | B | A | A | A |
| Example 1-24 | 2-13 | D-1 | C₆₀ | 1:1:1 | 1D | 1A | O atom | CN* | N | - | P | - | B | A | A | A |
| Example 1-25 | 2-14 | D-1 | C₆₀ | 1:1:1 | 1D | 2A | O atom | CN* | N | - | P | - | B | A | A | A |

a) Evaluation compound : p-type semiconductor : n-type semiconductor

From the results in Tble 1, it has been clarified that the photoelectric conversion element.

| Table 1 (Table 1-2) | Each component used to form photoelectric conversion film | | | | Remarks | | | | | | | | Evaluation result | | | |
|---|---|---|---|---|---|---|---|---|---|---|---|---|---|---|---|---|
| | Kind | | | Component ratio⁹ | Structure of D¹ | Structure of A¹ | Combination of Y¹¹ /Y¹¹ and Y¹¹/Y¹¹ | | Asymmetry of R^{d11}/R^{d12} | Structure of X^{d11}/R^{d22} | Presence of absence of F atom in D¹ or A¹ | Kind of A¹ | Evaluation 1 | Evaluation 2 | Evaluation 3 | Evaluation 4 |
| | Evaluation compound | p-type semiconductor material | n-type semiconductor material | | | | | | | | | | Photoelectric conversion efficiency after annealing | Variation of photoelectric conversion efficiency by annealing | Response speed | Electric field strength dependence of response speed |
| Example 1-26 | 2-15 | D-1 | C₆₀ | 1:1:1 | 1D | 2A | O atom | CN⁺ | N | - | P | - | B | A | A | A |
| Example 1-27 | 2-16 | D-1 | C₆₀ | 1:1:1 | 1D | 2A | O atom | CN⁺ | N | - | P | - | B | A | A | A |
| Example 1-28 | 2-17 | D-1 | C₆₀ | 1:1:1 | 1D | 2A | O atom | CN⁺ | N | - | P | - | B | A | A | A |
| Example 1-29 | 2-18 | D-1 | C₆₀ | 1:1:1 | 1D | 1A | O atom | CN⁺ | P | - | P | P | A | A | A | A |
| Example 1-30 | 2-19 | D-1 | C₆₀ | 1:1:1 | 1D | 1A | O atom | CN⁺ | P | - | P | P | AA | A | A | A |
| Example 1-31 | 2-20 | D-1 | C₆₀ | 1:1:1 | 1D | 1A | O atom | CN⁺ | P | - | P | P | A | A | B | A |
| Example 1-32 | 2-21 | D-1 | C₆₀ | 1:1:1 | 1D | 1A | O atom | S atom | N | - | P | - | B | A | B | A |
| Example 1-33 | 2-22 | D-1 | C₆₀ | 1:1:1 | 1D | 1A | O atom | CN⁺ | P | - | P | N | A | A | A | A |
| Example 1-34 | 2-23 | D-1 | C₆₀ | 1:1:1 | 1D | 1A | O atom | CN⁺ | P | - | P | P | AA | A | A | A |
| Example 1-35 | 2-24 | D-1 | C₆₀ | 1:1:1 | 1D | 1A | O atom | CN⁺ | P | - | P | N | A | A | A | A |
| Example 1-36 | 2-25 | D-1 | C₆₀ | 1:1:1 | 1D | 1A | O atom | CN⁺ | P | - | P | P | AA | A | A | A |
| Example 1-37 | 2-26 | D-1 | C₆₀ | 1:1:1 | 1D | 1A | O atom | CN⁺ | P | - | P | N | A | A | A | A |
| Example 1-38 | 2-27 | D-1 | C₆₀ | 1:1:1 | 1D | 1A | O atom | CN⁺ | P | - | P | P | AA | A | A | A |
| Example 1-39 | 2-28 | D-1 | C₆₀ | 1:1:1 | 1D | 1A | O atom | CN⁺ | P | - | P | N | A | A | A | A |
| Example 1-40 | 2-29 | D-1 | C₆₀ | 1:1:1 | 1D | 1A | O atom | CN⁺ | P | - | P | P | AA | A | A | A |
| Example 1-41 | 2-30 | D-1 | C₆₀ | 1:1:1 | 1D | 1A | O atom | CN⁺ | P | - | P | P | AA | A | A | A |
| Example 1-42 | 2-31 | D-1 | C₆₀ | 1:1:1 | 1D | 1A | O atom | CN⁺ | P | - | P | N | A | A | A | A |
| Example 1-43 | 2-32 | D-1 | C₆₀ | 1:1:1 | 1D | 1A | O atom | CN⁺ | P | - | P | P | AA | A | A | A |
| Comparative Example 1-1 | C1-1 | D-1 | C₆₀ | 1:1:1 | - | - | | - | - | - | - | - | D | B | D | D |
| Comparative Example 1-2 | C1-2 | D-1 | C₆₀ | 1:1:1 | - | - | | - | - | - | - | - | D | D | D | D |
| Comparative Example 1-3 | C1-3 | D-1 | C₆₀ | 1:1:1 | - | - | | - | - | - | - | - | D | D | D | D |
| Comparative Example 1-4 | C1-4 | D-1 | C₆₀ | 1:1:1 | - | - | | - | - | - | - | - | D | D | D | D |
| Comparative Example 1-5 | C1-5 | D-1 | C₆₀ | 1:1:1 | - | - | | - | - | - | - | - | D | D | D | D |
| Comparative Example 1-6 | C1-6 | D-1 | C₆₀ | 1:1:1 | - | - | | - | - | - | - | - | P | D | B | E |

a) Evaluation compound : p-type semiconductor : n-type semiconductor

in each of Examples had high photoelectric conversion efficiency in the visible light region (particularly, the wavelength range of 450 to 650 nm) even after the heat treatment (annealing treatment). In addition, it has been clarified that the photoelectric conversion element in each of Examples was excellent in the variation of the photoelectric conversion efficiency by the heat treatment (annealing treatment), the response speed, and the suppression of the electric field intensity dependence of the response speed.

From the comparison between Examples, it was confirmed that in a case where D¹ in the specific compound represented a group represented by Formula (1D), the variation in the photoelectric conversion efficiency by the heat treatment (annealing treatment), and the suppression of the electric field intensity dependence of the response speed were more excellent.

From the comparison between Examples (see Examples 1-1 to 1-10), it was confirmed that in a case where D¹ in the specific compound represented a group represented by Formula (2D), D¹ represented a group represented by Formula (6D), and at least one or more requirements X1 to X3 described above were satisfied, the photoelectric conversion efficiency by the heat treatment (annealing treatment), the variation of the photoelectric conversion efficiency by the heat treatment (annealing treatment), the response speed, and the suppression of the electric field intensity dependence of the response speed were more excellent.

From the comparison of Examples (see Examples 1-12 to 1-43), it was confirmed that in a case where D¹ in the specific compound represented a group represented by Formula (1D), and one of Y¹¹ or Y¹² in Formula (1A) represented by A¹ represented an oxygen atom and the other represented ═C(CN)₂ or one of Y²¹ or Y²² in Formula (2A) represented by A² represented an oxygen atom and the other represented ═C(CN)₂, the photoelectric conversion efficiency by the heat treatment (annealing treatment) and the response speed were more excellent.

From the comparison between Examples (see Examples 1-12 to 1-43), it was confirmed that in a case where D¹ in the specific compound represented a group represented by Formula (1D), and R^{d11} and R^{d12} in Formula (1D) had structures different from each other, the photoelectric conversion efficiency in the visible light region (particularly, the wavelength range of 450 to 650 nm) is higher even after the heat treatment (annealing treatment).

From the comparison between Examples (see Examples 1-12 to 1-43), it was confirmed that in a case where D¹ and A¹ in the specific compound did not have a fluorine atom, the response speed was more excellent.

In addition, from the comparison between Examples (see Examples 1-1 to 1-43), it was confirmed that in a case where A¹ in the specific compound was the group represented by Formula (1Aa) described above, X¹¹ represented —CR^{x}═, and X¹² represented a sulfur atom (except for the case where R^{x}’s in Formula (1Aa) are bonded to each other to form a ring), and a case where Y¹¹ represented an oxygen atom and Y¹² represents ═C(CN)₂ (specifically, a case where A¹ in the specific compound represented the group represented by Formula (1Aa-1) described above), the photoelectric conversion efficiency in the visible light region (particularly, the wavelength range of 450 to 650 nm) was more excellent even after the heat treatment (annealing treatment).

From the comparison between Examples (see Examples 1-29 and 1-30), it was confirmed that in a case where D¹ was the group represented by Formula (1D), one of R^{d11} or R^{d12} represented an alkyl group which may have a substituent (examples of the alkyl group include alkyl groups that are linear and branched, and a cycloalkyl group) and the other represented the group represented by Formula (X), the photoelectric conversion efficiency in the visible light region (particularly, the wavelength range of 450 to 650 nm) was more excellent even after the heat treatment (annealing treatment).

From the comparison between Examples (see Examples 1-30 and 1-31), it was confirmed that in a case where D¹ was the group represented by Formula (1D), one of R^{d11} or R^{d12} represented an alkyl group which may have a substituent (examples of the alkyl group include alkyl groups that are linear and branched, and a cycloalkyl group) and the other represented the group represented by Formula (ZB), the photoelectric conversion efficiency in the visible light region (particularly, the wavelength range of 450 to 650 nm) was more excellent and the response speed was more excellent even after the heat treatment (annealing treatment).

### Production of Photoelectric Conversion Element (B)

A photoelectric conversion element was produced by the same procedure as in the above-described [Production of Photoelectric Conversion Element (A)], except that each of materials (evaluation compounds, n-type semiconductor (fullerene (C₆₀)), p-type semiconductor, and coloring agent) illustrated in Table 2 was vapor-deposited to have a speed ratio illustrated in Table 2, thereby forming the photoelectric conversion film 12 having a bulk hetero structure.

### Evaluation of Photoelectric Conversion Element (B)

The photoelectric conversion element (B) was evaluated in the same manner as in [Evaluation of Photoelectric Conversion Element (A)]. The results are illustrated in Table 2. Here, in the evaluation of the photoelectric conversion element (B), Expression (S1) of the evaluation 1 and Expression (S3) of the evaluation 3 were obtained based on Expression described below.

$$\begin{matrix}\text{Relative photoelectric conversion efficiency = (integrated value of photoelectric} \\ \text{conversion efficiency of each photoelectric conversion element of Examples or Comparative} \\ \text{Examples in wavelength range of 450 to 650 nm after annealing)/(integrated value of} \\ \text{photoelectric conversion efficiency of photoelectric conversion element of Example 2-1 in} \\ \text{wavelength range of 450 to 650 nm after annealing)}\end{matrix} \qquad \text{Expression (S1):}$$

$$\begin{matrix}\text{Relative response speed = (rise time of each photoelectric conversion element of} \\ \text{Examples and Comparative Examples)/(rise time of photoelectric conversion element of} \\ \text{Example 2-1)}\end{matrix} \qquad \text{Expression (S3):}$$

TABLE 3

| Table 2 (Table 2-1) | Each component used to form photoelectric conversion film | | | | | Remarks | | | | | | | | Evaluation results | | | |
|---|---|---|---|---|---|---|---|---|---|---|---|---|---|---|---|---|---|
| | Kind | | | | Component ratio² | Structureof D¹ | Structure of A¹ | Combination of Y¹¹/Y¹² and Y²¹/Y²² | | Asymmetry of R^{d11}/R^{d12} | Structure of X^{d21}/R^{d22} | Presence or absence of F atom in D¹ or A¹ | Kind of A¹ | Evaluation 1 | Evaluation 2 | Evaluation 3 | Evaluation 4 |
| | Evalutation compound | Coloring agent | p-type semiconductor material | n-type semiconductor material | | | | | | | | | | Photoelectric conversion efficiency after annealing | Variation of photoelectric conversion efficiency by annealing | Response speed | Electric field strength dependence of response speed |
| Example 2-1 | 1-1 | B-1 | D-1 | C₆₀ | 0.5:0.5:1:1 | 2D | 1A | O atom | CN* | - | N | P | N | C | C | C | C |
| Example 2-2 | 1-1 | B-2 | D-1 | C₆₀ | 0.5:0.5:1:1 | 2D | 1A | O atom | CN* | - | N | P | N | B | C | C | C |
| Example 2-3 | 1-1 | B-3 | D-1 | C₆₀ | 0.5:0.5:1:1 | 2D | 1A | O atom | CN* | - | N | P | N | B | C | C | C |
| Example 2-4 | 1-1 | B-4 | D-1 | C₆₀ | 0.5:0.5:1:1 | 2D | 1A | O atom | CN* | - | N | P | N | B | C | C | C |
| Example 2-5 | 1-1 | B-5 | D-1 | C₆₀ | 0.5:0.5:1:1 | 2D | 1A | O atom | CN* | - | N | P | N | B | C | C | C |
| Example 2-6 | 1-1 | B-6 | D-1 | C₆₀ | 0.5:0.5:1:1 | 2D | 1A | O atom | CN* | - | N | P | N | B | C | C | C |
| Example 2-7 | 1-1 | B-7 | D-1 | C₆₀ | 0.5:0.5:1:1 | 2D | 1A | O atom | CN* | - | N | P | N | B | C | C | C |
| Example 2-8 | 1-2 | B-7 | D-1 | C₆₀ | 0.5:0.5:1:1 | 2D | 1A | O atom | CN* | - | N | P | P | A | C | B | C |
| Example 2-9 | 1-3 | B-7 | D-1 | C₆₀ | 0.5:0.5:1:1 | 2D | 1A | O atom | CN* | - | N | P | N | B | C | C | C |
| Example 2-10 | 1-4 | B-7 | D-1 | C₆₀ | 0.5:0.5:1:1 | 2D | 1A | O atom | CN* | - | N | P | P | A | C | B | C |
| Example 2-11 | 1-5 | B-7 | D-1 | C₆₀ | 0.5:0.5:1:1 | 2D | 1A | O atom | CN* | - | P | P | P | AA | B | A | B |
| Example 2-12 | 1-6 | B-7 | D-1 | C₆₀ | 0.5:0.5:1:1 | 2D | 1A | O atom | CN* | - | P | P | P | AA | B | A | B |
| Example 2-13 | 1-7 | B-7 | D-1 | C₆₀ | 0.5:0.5:1:1 | 2D | 1A | O atom | CN* | - | P | P | P | AA | B | A | B |
| Example 2-14 | 1-8 | B-7 | D-1 | C₆₀ | 0.5:0.5:1:1 | 2D | 1A | O atom | CN* | - | P | P | P | AA | B | A | B |
| Example 2-15 | 1-9 | B-7 | D-1 | C₆₀ | 0.5:0.5:1:1 | 2D | 1A | O atom | CN* | - | N | P | P | A | C | B | C |
| Example 2-16 | 1-10 | B-7 | D-1 | C₆₀ | 0.5:0.5:1:1 | N | 1A | O atom | CN* | - | - | P | P | A | C | B | C |
| Example 2-23 | 2-1 | B-7 | D-1 | C₆₀ | 0.5:0.5:1:1 | 1D | 1A | O atom | CN* | N | - | P | N | A | A | A | A |
| Example 2-24 | 2-2 | B-7 | D-1 | C₆₀ | 0.5:0.5:1:1 | 1D | 1A | O atom | CN* | N | - | P | P | AA | A | A | A |
| Example 2-25 | 2-3 | B-7 | D-1 | C₆₀ | 0.5:0.5:1:1 | 1D | 1A | O atom | CN* | N | - | N | P | A | A | B | A |
| Example 2-26 | 2-4 | B-7 | D-1 | C₆₀ | 0.5:0.5:1:1 | 1D | 1A | O atom | CN* | N | - | P | P | AA | A | A | A |
| Example 2-27 | 2-5 | B-7 | D-1 | C₆₀ | 0.5:0.5:1:1 | 1D | 1A | O atom | CN* | N | - | P | P | AA | A | A | A |
| Example 2-28 | 2-6 | B-7 | D-1 | C₆₀ | 0.5:0.5:1:1 | 1D | 1A | O atom | CN* | N | - | P | N | AA | A | A | A |
| Example 2-29 | 2-7 | B-7 | D-1 | C₆₀ | 0.5:0.5:1:1 | 1D | 1A | O atom | CN* | N | - | P | P | AA | A | A | A |
| Example 2-30 | 2-8 | B-7 | D-1 | C₆₀ | 0.5:0.5:1:1 | 1D | 1A | O atom | CN* | N | - | N | N | A | A | B | A |

a) Evaluation compound : p-type Semiconductor : n-type Semiconductor

TABLE 4

| Table 2 (Table 2-2) | Each component used to form photoelectric conversion film | | | | | Remarks | | | | | | | | Evaluation result | | | |
|---|---|---|---|---|---|---|---|---|---|---|---|---|---|---|---|---|---|
| | Kind | | | | Component ratio | Structure of D¹ | Structure of A¹ | Combination of Y and Y²¹/Y²² | | Asymmetry of R | Structure of X | Presence or absence of atom in D¹ or A¹ | Kind of A¹ | Evaluation 1 | Evaluation 2 | Evaluation 3 | Evaluation 4 |
| | Evaluation compound | Coloring agent | p-type semiconductor material | n-type semiconductor material | | | | | | | | | | Photoelectric conversion efficiency after annealing | Variation of photoelectric conversion efficiency by annealing | Response speed | Electric field strength dependence of response speed |
| Example 2-31 | 2-9 | B-7 | D-1 | C₆₀ | 0.5:0.5:1:1 | 1D | 1A | O atom | CN* | N | - | N | P | A | A | B | A |
| Example 2-32 | 2-10 | B-7 | D-1 | C₆₀ | 0.5:0.5:1:1 | 1D | 1A | O atom | CN* | N | - | P | - | A | A | A | A |
| Example 2-33 | 2-11 | B-7 | D-1 | C₆₀ | 0.5:0.5:1:1 | 1D | 1A | O atom | CN* | N | - | P | - | A | A | A | A |
| Example 2-34 | 2-12 | B-7 | D-1 | C₆₀ | 0.5:0.5:1:1 | 1D | 1A | O atom | CN* | N | - | P | - | A | A | A | A |
| Example 2-35 | 2-13 | B-7 | D-1 | C₆₀ | 0.5:0.5:1.1 | 1D | 1A | O atom | CN* | N | - | P | - | A | A | A | A |
| Example 2-36 | 2-14 | B-7 | D-1 | C₆₀ | 0.5:0.5:1.1 | 1D | 2A | O atom | CN* | N | - | P | - | A | A | A | A |
| Example 2-37 | 2-15 | B-7 | D-1 | C₆₀ | 0.5:0.5:1:1 | 1D | 2A | O atom | CN* | N | - | P | - | A | A | A | A |
| Example 2-38 | 2-16 | B-7 | D-1 | C₆₆ | 0.5:0.5:1:1 | 1D | 2A | Oatom | CN* | N | - | P | - | A | A | A | A |
| Example 2-39 | 2-17 | B-7 | D-1 | C₆₀ | 0.5:0.5:1:1 | 1D | 2A | O atom | CN* | N | - | P | - | A | A | A | A |
| Example 2-40 | 2-18 | B-7 | D-1 | C₆₀ | 0.5:0.5:1:1 I: | 1D | 1A | O atom | CN* | P | - | P | P | AA | A | A | A |
| Example 2-41 | 2-19 | B-7 | D-1 | C₆₆ | 0.5:0.5:1:1 I | 1D | 1A | O atom | CN* | P | - | P | P | AA | A | A | A |
| Example 2-42 | 2-20 | B-7 | D-1 | C₆₆ | 0.5:0.5:1:1 | 1D | 1A | O αtom | CN* | P | - | P | P | AA | A | B | A |
| Example 2-43 | 2-21 | B-7 | D-1 | C₆₆ | 0.5:0.5:1:1 | 1D | 1A | O atom | S atom | N | - | P | - | A | A | B | A |
| Example 2-44 | 2-22 | B-7 | D-1 | C₆₆ | 0.5.0.5:1:1 1.1 | 1D | 1A | O atom | CN* | P | - | P | N | AA | A | A | A |
| Example 2-45 | 2-23 | B-7 | D-1 | C₆₆ | 0.5:0.5:1:1 | 1D | 1A | O atom | CN* | P | - | P | P | AA | A | A | A |
| Example 2-46 | 2-24 | B-7 | D-1 | C₆₆ | 0.50.5:1:1 | 1D | 1A | O atom | CN* | P | - | P | N | AA | A | A | A |
| Example 2-17 | 2-25 | B-7 | D-1 | C₆₆ | 0.5:0.5:1:1 | 1D | 1A | O atom | CN* | P | - | P | P | AA | A | A | A |
| Example 2-48 | 2-26 | B-7 | D-1 | C₆₆ | 0.5:0.5:1:1 | 1D | 1A | O atom | CN* | P | - | P | N | AA | A | A | A |
| Example 2-49 | 2-27 | B-7 | D-1 | C₆₆ | 0.5:0.5:1:1 | 1D | 1A | O atom | CN* | P | - | P | P | AA | A | A | A |
| Example 2.50 | 2-28 | B-7 | D-1 | C₆₀ | 0.5:0.5:1:1 | 1D | 1A | O atom | CN* | P | - | P | N | AA | A | A | A |
| Fxample 2-51 | 2-29 | B-7 | D-1 | C₆₆ | 0.5:0.5:1:1 | 1D | 1A | O atom | CN* | P | - | P | P | AA | A | A | A |
| Example 2-52 | 2-30 | B-7 | D-1 | C₆₆ | 0.5:0.5:1:1 | 1D | 1A | O atom | CN* | P | - | P | P | AA | A | A | A |
| Example 2-53 | 2-31 | B-7 | D-1 | C₆₀ | 0.5:0.5:1:1 | 1D | 1A | O atom | CN* | P | - | P | N | AA | 1A | A | A |
| Example 2-54 | 2-32 | B-7 | D-1 | C₆₀ | 0.5:0.5:1:1 | 1D | 1A | O atom | CN* | P | - | P | P | AA | A | A | A |
| Comparative Example 2-1 | Cl-1 | B-7 | D-1 | C₆₀ | 0.5:0.5:1:1 | - | - | - | - | - | - | - | - | C | E | D | D |
| Comparative Example 2-2 | Cl-2 | B-7 | D-1 | C₆₀ | 0.5:0.5:1:1 | - | - | - | - | - | - | - | - | C | D | D | D |
| Comparative Example 2-3 | Cl-3 | B-7 | D-1 | C₆₀ | 0.5:0.5:1:1 | - | - | - | - | - | - | - | - | C | D | D | D |
| Comparative Example 2-4 | Cl-4 | B-7 | D-1 | C₆₀ | 0.5:0.5:1:1 | - | - | - | - | - | - | - | - | C | D | D | D |
| Comparative Example 2-5 | Cl-5 | B-7 | D-1 | C₆₀ | 0.5:0.5:1:1 | - | - | - | - | - | - | - | - | C | D | D | D |
| Comparative Example 2-6 | Cl-6 | B-7 | D-1 | C₆₀ | 0.5:0.5:1:1 | - | - | - | - | - | - | - | - | D | D | E | E |

a) Evatuation compound : p-type semiconductor : n-type semiconductor

in each of Examples had high photoelectric conversion efficiency in the visible light region (particularly, the wavelength range of 450 to 650 nm) even after the heat treatment (annealing treatment). In addition, it has been clarified that the photoelectric conversion element in each of Examples was excellent in the variation of the photoelectric conversion efficiency by the heat treatment (annealing treatment), the response speed, and the suppression of the electric field intensity dependence of the response speed.

From the comparison between Examples, it was confirmed that in a case where D¹ in the specific compound represented a group represented by Formula (1D), the variation in the photoelectric conversion efficiency by the heat treatment (annealing treatment), and the suppression of the electric field intensity dependence of the response speed were more excellent.

From the comparison of Examples (see Examples 2-23 to 2-54), it was confirmed that in a case where D¹ in the specific compound represented a group represented by Formula (1D), and one of Y¹¹ or Y¹² in Formula (1A) represented by A¹ represented an oxygen atom and the other represented ═C(CN)₂ or one of Y²¹ or Y²² in Formula (2A) represented by A² represented an oxygen atom and the other represented ═C(CN)₂, the photoelectric conversion efficiency by the heat treatment (annealing treatment) and the response speed were more excellent.

From the comparison between Examples (see Examples 2-23 to 2-54), it was confirmed that in a case where D¹ in the specific compound represented a group represented by Formula (1D), and R^{d11} and R^{d12} in Formula (1D) had structures different from each other, the photoelectric conversion efficiency in the visible light region (particularly, the wavelength range of 450 to 650 nm) is higher even after the heat treatment (annealing treatment).

From the comparison between Examples (see Examples 2-23 to 2-54), it was confirmed that in a case where D¹ and A¹ in the specific compound did not have a fluorine atom, the response speed was more excellent.

From the comparison between Examples (see Examples 2-41 and 2-42), it was confirmed that in a case where D¹ was the group represented by Formula (1D), one of R^{d11} or Rd¹² represented an alkyl group which may have a substituent (examples of the alkyl group include alkyl groups that are linear and branched, and a cycloalkyl group) and the other represented the group represented by Formula (ZB), the response speed was more excellent.

### Explanation of References

10a, 10b: photoelectric conversion element
11: conductive film (lower electrode)
12: photoelectric conversion film
15: transparent conductive film (upper electrode)
16A: electron blocking film
16B: positive hole blocking film

What is claimed is:

1. A photoelectric conversion element comprising, in the following order:
a conductive film;
a photoelectric conversion film; and
a transparent conductive film,
wherein the photoelectric conversion film at least contains a compound represented by Formula (1), 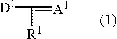 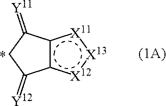 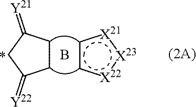
in Formula (1), A¹ represents a group represented by any one of Formula (1A) or Formula (2A),
D¹ represents a substituent having a nitrogen atom and an aromatic ring,
R¹ represents a hydrogen atom or a substituent,
in Formula (1A), Y¹¹ and Y¹² each independently represent an oxygen atom, a sulfur atom, ═C(CN)₂, =C(COR¹¹)₂, ═C(SO₂R¹¹)₂, =C(SOR¹¹)₂, =C(CN)(COR¹¹), ═C(CN)(SO₂R¹¹), =C(CN)(SOR¹¹), =C(COR¹¹)(SO₂R¹¹), =C(COR¹¹)(SOR¹¹), or =C(SO₂R¹¹)(SOR¹¹), R¹¹ each independently represents a hydrogen atom, a hydrocarbon group which may have a substituent, or a heteroaryl group which may have a substituent, where Y¹¹ and Y¹² are not the same as each other,
one of X¹¹ or X¹² represents a sulfur atom, an oxygen atom, a selenium atom, or NR¹², and the other represents a nitrogen atom or —CR^{x}═, X¹³ represents —CR^{x}═ or a nitrogen atom, R¹² represents a hydrogen atom or a substituent, R^{x} represents a hydrogen atom or a substituent, in a case where X¹¹ and X¹³ each represent —CR^{x}═, R^{x}’s may be bonded to each other to form a ring, in a case where X¹² and X¹³ each represent —CR^{x}═, R^{x}’s may be bonded to each other to form a ring,
a dotted line specified in Formula (1A) represents that a ring structure having X¹¹ to X¹³ and carbon atoms respectively adjacent to X¹¹ and X¹³ in Formula (1A) as ring member atoms forms a resonance structure,
* represents a bonding position,
in Formula (2A), Y²¹ and Y²² each independently represent an oxygen atom, a sulfur atom, ═C(CN)₂, =C(COR²¹)₂, ═C(SO₂R²¹)₂, =C(SOR²¹)₂, =C(CN)(COR²¹), ═C(CN)(SO₂R²¹), =C(CN)(SOR²¹), =C(COR²¹)(SO₂R²¹), =C(COR²¹)(SOR²¹), or =C(SO₂R²¹)(SOR²¹), R²¹ each independently represents a hydrogen atom, a hydrocarbon group which may have a substituent, or a heteroaryl group which may have a substituent, where Y²¹ and Y²² are not the same as each other,
B represents a 5- or 6-membered monocyclic aromatic ring which may have a substituent,
one of X²¹ or X²² represents a sulfur atom, an oxygen atom, a selenium atom, or NR²², and the other represents a nitrogen atom or -CR^{Y}=, X²³ represents -CR^{Y}= or a nitrogen atom, R²² represents a hydrogen atom or a substituent, R^{Y} represents a hydrogen atom or a substituent, in a case where X²¹ and X²³ each represent -CR^{Y}=, R^{y}’s may be bonded to each other to form a ring, in a case where X²² and X²³ each represent -CR^{Y}=, R^{y}’s may be bonded to each other to form a ring,
a dotted line specified in Formula (2A) represents that a ring structure having X²¹ to X²³ and carbon atoms respectively adjacent to X²¹ and X²³ in Formula (2A) as ring member atoms forms a resonance structure, and
* represents a bonding position.

2. The photoelectric conversion element according to claim 1,
wherein one of Y¹¹ or Y¹² represents an oxygen atom or a sulfur atom, and
one of Y²¹ or Y²² represents an oxygen atom or a sulfur atom.

3. The photoelectric conversion element according to claim 1,
wherein one of Y¹¹ or Y¹² represents ═C(CN)₂, and
one of Y2¹ or Y²² represents ═C(CN)₂.

4. The photoelectric conversion element according to claim 1,
wherein A¹ represents a group represented by Formula (1Aa-1), 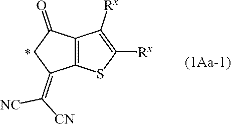
in Formula (1Aa-1), R
^{x} represents a hydrogen atom or a substituent.

5. The photoelectric conversion element according to claim 1,
wherein D¹ represents a group represented by any one of Formula (1D) or Formula (2D), 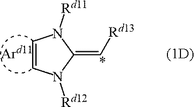 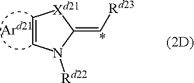
in Formula (1D), * represents a bonding position,
Ar^{d11} represents an aromatic ring containing two or more carbon atoms, which may have a substituent,
R^{d11} and R^{d12} each independently represent an aromatic ring group or -C(R^{L11})(R^{L12})(R^{L13}), which may have a substituent, R^{L11} to R^{L13} each independently represent a hydrogen atom, an alkyl group which may have a substituent, or an aromatic ring group which may have a substituent, R^{L11} to R^{L13} may be bonded to each other to form a ring,
R^{d13} represents a hydrogen atom or a substituent,
in Formula (2D), * represents a bonding position,
Ar^{d21} represents an aromatic ring containing two or more carbon atoms, which may have a substituent,
R^{d22} represents an aromatic ring group which may have a substituent or -C(R^{L21})(R^{L22})(R^{L23}), R^{L21} to R^{L23} each independently represent a hydrogen atom, an alkyl group which may have a substituent, or an aromatic ring group which may have a substituent, R^{L21} to R^{L23} may be bonded to each other to form a ring,
X^{d21} represents a sulfur atom, an oxygen atom, or -C(R^{L24})(R^{L25})-, R^{L24} and R^{L25} each independently represent a hydrogen atom, an alkyl group which may have a substituent, or an aromatic ring group which may have a substituent, R^{L24} and R^{L25} may be bonded to each other to form a ring, and
R^{d23} represents a hydrogen atom or a substituent.

6. The photoelectric conversion element according to claim 5,
wherein D¹ represents a group represented by Formula (1D).

7. The photoelectric conversion element according to claim 6,
wherein the group represented by Formula (1D) represents a group represented by Formula (3D), 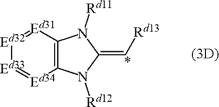
in Formula (3D), * represents a bonding position,
R^{d11} and R^{d12} each independently represent an aromatic ring group or -C(R^{L11})(R^{L12})(R^{L13}), which may have a substituent, R^{L11} to R^{L13} each independently represent a hydrogen atom, an alkyl group which may have a substituent, or an aromatic ring group which may have a substituent, R^{L11} to R^{L13} may be bonded to each other to form a ring,
R^{d13} represents a hydrogen atom or a substituent,
E^{d31} to E^{d34} each independently represent a nitrogen atom or -CR^{E31}=, R^{E31} represents a hydrogen atom or a substituent, and in a case where a plurality of R^{E31}′s exist, R^{E31}′s may be bonded to each other to form a ring.

8. The photoelectric conversion element according to claim 6,
wherein the group represented by Formula (1D) represents a group represented by Formula (4D), 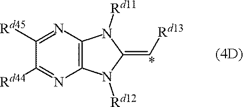
in Formula (4D), * represents a bonding position,
R^{d11} and R^{d12} each independently represent an aromatic ring group or -C(R^{L11})(R^{L12})(R^{L13}), which may have a substituent, R^{L11} to R^{L13} each independently represent a hydrogen atom, an alkyl group which may have a substituent, or an aromatic ring group which may have a substituent, R^{L11} to R^{L13} may be bonded to each other to form a ring,
R^{dl3}, R^{d44}, and R^{d45} each independently represent a hydrogen atom or a substituent, and R^{d44} and R^{d45} may be bonded to each other to form a ring.

9. The photoelectric conversion element according to claim 6,
wherein the group represented by Formula (1D) is a group represented by Formula (5D), 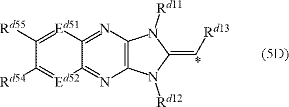
in Formula (5D), * represents a bonding position,
R^{d11} and R^{d12} each independently represent an aromatic ring group or -C(R^{L11})(R^{L12})(R^{L13}), which may have a substituent, R^{L11} to R^{L13} each independently represent a hydrogen atom, an alkyl group which may have a substituent, or an aromatic ring group which may have a substituent, R^{L11} to R^{L13} may be bonded to each other to form a ring,
R^{d13} R^{d54}, and R^{d55} each independently represent a hydrogen atom or a substituent, R^{d54} and R^{d55} may be bonded to each other to form a ring,
E^{d51} and E^{d52} each independently represent a nitrogen atom or -CR^{E51}=, and R^{E51} represents a hydrogen atom or a substituent.

10. The photoelectric conversion element according to claim 5,
wherein structures of R^{d11} and R^{d12} are different from each other.

11. The photoelectric conversion element according to claim 5,
wherein one of R^{d11} or R^{d12} represents an alkyl group which may have a substituent, and the other represents a group represented by Formula (X), 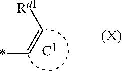
in Formula (X), * represents a bonding position, C¹ represents a monocyclic aromatic ring containing at least two carbon atoms, which may have a substituent other than R^{d1}, and R^{d1} represents an alkyl group which may have a substituent, a silyl group which may have a substituent, an alkoxy group which may have a substituent, an alkylthio group which may have a substituent, a cyano group, a halogen atom, an aryl group which may have a substituent, a heteroaryl group which may have a substituent, an alkenyl group which may have a substituent, or an alkynyl group which may have a substituent.

12. The photoelectric conversion element according to claim 11,
wherein the group represented by Formula (X) represents a group represented by Formula (ZB), 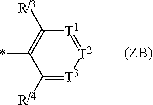
in Formula (ZB), T¹ to T³ each independently represent —CR^{e12}═ or a nitrogen atom, R^{e12} represents a hydrogen atom or a substituent, R^{f3} and R^{f4} each independently represent an alkyl group which may have a substituent, an aryl group which may have a substituent, or a heteroaryl group which may have a substituent, and * represents a bonding position.

13. The photoelectric conversion element according to claim 5,
wherein D¹ represents a group represented by Formula (2D), and
the group represented by Formula (2D) represents a group represented by Formula (6D), 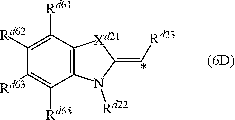
in Formula (6D), * represents a bonding position,
R^{d22} represents an aromatic ring group which may have a substituent or -C(R^{L21})(R^{L22})(R^{L23}), R^{L21} to R^{L23} each independently represent a hydrogen atom, an alkyl group which may have a substituent, or an aromatic ring group which may have a substituent, R^{L21} to R^{L23} may be bonded to each other to form a ring,
X^{d21} represents a sulfur atom, an oxygen atom, or -C(R^{L24})(R^{L25})-, R^{L24} and R^{L25} each independently represent a hydrogen atom, an alkyl group which may have a substituent, or an aromatic ring group which may have a substituent, R^{L24} and R^{L25} may be bonded to each other to form a ring,
R^{d23} and R^{d61} to R^{d64} each independently represent a hydrogen atom or a substituent, and R^{d61} to R^{d64} may be bonded to each other to form a ring.

14. The photoelectric conversion element according to claim 13,
wherein Formula (6D) satisfies at least one or more following requirements,
a requirement X1: R^{d22} represents an aromatic ring group which may have a substituent,
a requirement X2: X^{d21} represents -C(R^{L24})(R^{L25})-, and R^{L24} and R^{L25} are bonded to each other to form a ring, and
a requirement X3: any one or more of R^{d61} and R^{d62}, R^{d62} and R^{d63}, and R^{d63} and R^{d64} are bonded to each other to form a ring.

15. The photoelectric conversion element according to claim 1,
wherein the photoelectric conversion film further contains a n-type semiconductor.

16. The photoelectric conversion element according to claim 15,
wherein the n-type semiconductor includes fullerenes selected from the group consisting of a fullerene and a derivative thereof.

17. The photoelectric conversion element according to claim 1,
wherein the photoelectric conversion film further contains a p-type semiconductor.

18. The photoelectric conversion element according to claim 1, further comprising one or more interlayers between the conductive film and the transparent conductive film, in addition to the photoelectric conversion film.

19. An imaging element comprising the photoelectric conversion element according to claim 1.

20. An optical sensor comprising the photoelectric conversion element according to claim 1.

21. A compound represented by Formula (1), 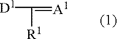 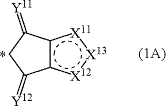 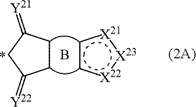
in Formula (1), A¹ represents a group represented by any one of Formula (1A) or Formula (2A),
D¹ represents a substituent having a nitrogen atom and an aromatic ring,
R¹ represents a hydrogen atom or a substituent,
in Formula (1A), Y¹¹ and Y¹² each independently represent an oxygen atom, a sulfur atom, ═C(CN)₂, =C(COR¹¹)₂, ═C(SO₂R¹¹)₂, =C(SOR¹¹)₂, =C(CN)(COR¹¹), ═C(CN)(SO₂R¹¹), =C(CN)(SOR¹¹), =C(COR¹¹)(SO₂R¹¹, =C(COR¹¹)(SOR¹¹), or =C(SO₂R¹¹)(SOR¹¹), R¹¹ each independently represents a hydrogen atom, a hydrocarbon group which may have a substituent, or a heteroaryl group which may have a substituent, where Y¹¹ and Y¹² are not the same as each other,
one of X¹¹ or X¹² represents a sulfur atom, an oxygen atom, a selenium atom, or NR¹², and the other represents a nitrogen atom or —CR^{x}═, X¹³ represents —CR^{x}═ or a nitrogen atom, R¹² represents a hydrogen atom or a substituent, R^{x} represents a hydrogen atom or a substituent, in a case where X¹¹ and X¹³ each represent —CR^{x}═, R^{x}’s may be bonded to each other to form a ring, in a case where X¹² and X¹³ each represent —CR^{x}═, R^{x}’s may be bonded to each other to form a ring,
a dotted line specified in Formula (1A) represents that a ring structure having X¹¹ to X¹³ and carbon atoms respectively adjacent to X¹¹ and X¹³ in Formula (1A) as ring member atoms forms a resonance structure,
* represents a bonding position,
in Formula (2A), Y²¹ and Y²² each independently represent an oxygen atom, a sulfur atom, ═C(CN)₂, =C(COR²¹)₂, ═C(SO₂R²¹)₂, =C(SOR²¹)₂, =C(CN)(COR²¹), ═C(CN)(SO₂R²¹), =C(CN)(SOR²¹), =C(COR²¹)(SO₂R²¹), =C(COR²¹)(SOR²¹), or =C(SO₂R²¹)(SOR²¹), R²¹ each independently represents a hydrogen atom, a hydrocarbon group which may have a substituent, or a heteroaryl group which may have a substituent, where Y²¹ and Y²² are not the same as each other,
B represents a 5- or 6-membered monocyclic aromatic ring which may have a substituent,
one of X²¹ or X²² represents a sulfur atom, an oxygen atom, a selenium atom, or NR²², and the other represents a nitrogen atom or -CR^{Y}=, X²³ represents -CR^{Y}= or a nitrogen atom, R²² represents a hydrogen atom or a substituent, R^{y} represents a hydrogen atom or a substituent, in a case where X²¹ and X²³ each represent —CR^{y}═, R^{y}’s may be bonded to each other to form a ring, in a case where X²² and X²³ each represent -CR^{Y}=, R^{y}’s may be bonded to each other to form a ring,
a dotted line specified in Formula (2A) represents that a ring structure having X²¹ to X²³ and carbon atoms respectively adjacent to X²¹ and X²³ in Formula (2A) as ring member atoms forms a resonance structure, and
* represents a bonding position.

22. The compound according to claim 21,
wherein one of Y¹¹ or Y¹² represents an oxygen atom or a sulfur atom, and
one of Y2¹ or Y²² represents an oxygen atom or a sulfur atom.

23. The compound according to claim 21,
wherein one of Y¹¹ or Y¹² represents ═C(CN)₂, and
one of Y²¹ or Y²² represents ═C(CN)₂.

24. The compound according to claim 21,
wherein A¹ represents a group represented by Formula (1Aa-1), 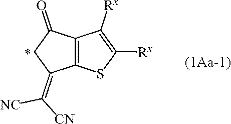
in Formula (1Aa-1), R^{x} represents a hydrogen atom or a substituent.

25. The compound according to claim 21,
wherein D¹ represents a group represented by any one of Formula (1D) or Formula (2D), 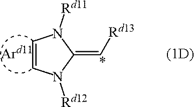 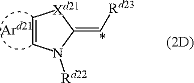
in Formula (1D), * represents a bonding position,
Ar^{d11} represents an aromatic ring containing two or more carbon atoms, which may have a substituent,
R^{d11} and R^{d12} each independently represent an aromatic ring group or -C(R^{L11})(R^{L12})(R^{L13}), which may have a substituent, R^{L11} to R^{L13} each independently represent a hydrogen atom, an alkyl group which may have a substituent, or an aromatic ring group which may have a substituent, R^{L11} to R^{L13} may be bonded to each other to form a ring,
R^{d13} represents a hydrogen atom or a substituent,
in Formula (2D), * represents a bonding position,
Ar^{d21} represents an aromatic ring containing two or more carbon atoms, which may have a substituent,
R^{d22} represents an aromatic ring group which may have a substituent or -C(R^{L21})(R^{L22})(R^{L23}), R^{L21} to R^{L23} each independently represent a hydrogen atom, an alkyl group which may have a substituent, or an aromatic ring group which may have a substituent, R^{L21} to R^{L23} may be bonded to each other to form a ring,
X^{d21} represents a sulfur atom, an oxygen atom, or -C(R^{L24})(R^{L25})-, R^{L24} and R^{L25} each independently represent a hydrogen atom, an alkyl group which may have a substituent, or an aromatic ring group which may have a substituent, R^{L24} and R^{L25} may be bonded to each other to form a ring,
R^{d23} represents a hydrogen atom or a substituent.

26. The compound according to claim 25,
wherein D¹ represents a group represented by Formula (1D).

27. The compound according to claim 26,
wherein the group represented by Formula (1D) represents a group represented by Formula (3D), 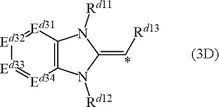
in Formula (3D), * represents a bonding position,
R^{d11} and R^{d12} each independently represent an aromatic ring group or -C(R^{L11})(R^{L12})(R^{L13}), which may have a substituent, R^{L11} to R^{L13} each independently represent a hydrogen atom, an alkyl group which may have a substituent, or an aromatic ring group which may have a substituent, R^{L11} to R^{L13} may be bonded to each other to form a ring,
R^{d13} represents a hydrogen atom or a substituent,
E^{d31} to E^{d34} each independently represent a nitrogen atom or -CR^{E31}=, R^{E31} represents a hydrogen atom or a substituent, and in a case where a plurality of R^{E31}′s exist, R^{E31}′s may be bonded to each other to form a ring.

28. The compound according to claim 26,
wherein the group represented by Formula (1D) represents a group represented by Formula (4D), 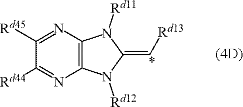
in Formula (4D), * represents a bonding position,
R^{d11} and R^{d12} each independently represent an aromatic ring group or -C(R^{L11})(R^{L12})(R^{L13}), which may have a substituent, R^{L11} to R^{L13} each independently represent a hydrogen atom, an alkyl group which may have a substituent, or an aromatic ring group which may have a substituent, R^{L11} to R^{L13} may be bonded to each other to form a ring,
R^{d13}, R^{d44}, and R^{d45} each independently represent a hydrogen atom or a substituent, and R^{d44} and R^{d45} may be bonded to each other to form a ring.

29. The compound according to claim 26,
wherein the group represented by Formula (1D) is a group represented by Formula (5D), 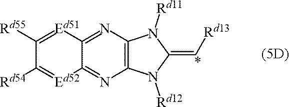
in Formula (5D), * represents a bonding position,
R^{d11} and R^{d12} each independently represent an aromatic ring group or -C(R^{L11})(R^{L12})(R^{L13}), which may have a substituent, R^{L11} to R^{L13} each independently represent a hydrogen atom, an alkyl group which may have a substituent, or an aromatic ring group which may have a substituent, R^{L11} to R^{L13} may be bonded to each other to form a ring,
R⁴³ R^{d54}, and R^{d55} each independently represent a hydrogen atom or a substituent, R^{d54} and R^{d55} may be bonded to each other to form a ring,
E^{d51} and E^{d52} each independently represent a nitrogen atom or -CR^{E51}=, and R^{E51} represents a hydrogen atom or a substituent.

30. The compound according to claim 25,
wherein structures of R^{d11} and R^{d12} are different from each other.

31. The compound according to claim 25,
wherein one of R^{d11} or R^{d12} represents an alkyl group which may have a substituent, and the other represents a group represented by Formula (X), 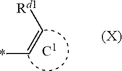
in Formula (X), * represents a bonding position, C¹ represents a monocyclic aromatic ring containing at least two carbon atoms, which may have a substituent other than R^{d1}, and R^{d1} represents an alkyl group which may have a substituent, a silyl group which may have a substituent, an alkoxy group which may have a substituent, an alkylthio group which may have a substituent, a cyano group, a halogen atom, an aryl group which may have a substituent, a heteroaryl group which may have a substituent, an alkenyl group which may have a substituent, or an alkynyl group which may have a substituent.

32. The compound according to claim 31,
wherein the group represented by Formula (X) represents a group represented by Formula (ZB), 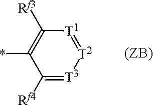
in Formula (ZB), T¹ to T³ each independently represent —CR^{e12}═ or a nitrogen atom, R^{e12} represents a hydrogen atom or a substituent, R^{f3} and R^{f4} each independently represent an alkyl group which may have a substituent, an aryl group which may have a substituent, or a heteroaryl group which may have a substituent, and * represents a bonding position.

33. The compound according to claim 25,
wherein D¹ represents a group represented by Formula (2D), and
the group represented by Formula (2D) represents a group represented by Formula (6D), 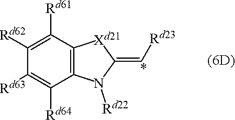
in Formula (6D), * represents a bonding position,
R^{d22} represents an aromatic ring group which may have a substituent or -C(R^{L21})(R^{L22})(R^{L23}), R^{L21} to R^{L23} each independently represent a hydrogen atom, an alkyl group which may have a substituent, or an aromatic ring group which may have a substituent, R^{L21} to R^{L23} may be bonded to each other to form a ring,
X^{d21} represents a sulfur atom, an oxygen atom, or -C(R^{L24})(R^{L25})-, R^{L24} and R^{L25} each independently represent a hydrogen atom, an alkyl group which may have a substituent, or an aromatic ring group which may have a substituent, R^{L24} and R^{L25} may be bonded to each other to form a ring,
R^{d23} and R^{d61} to R^{d64} each independently represent a hydrogen atom or a substituent, and R^{d61} to R^{d64} may be bonded to each other to form a ring.

34. The compound according to claim 33,
wherein Formula (6D) satisfies at least one or more following requirements,
a requirement X1: R^{d22} represents an aromatic ring group which may have a substituent,
a requirement X2: X^{d21} represents -C(R^{L24})(R^{L25})-, and R^{L24} and R^{L25} are bonded to each other to form a ring, and
a requirement X3: any one or more of R^{d61} and R^{d62}, R^{d62} and R^{d63}, and R^{d63} and R^{d64} are bonded to each other to form a ring.
